(19) United States
(12) Patent Application Publication
(10) Pub. No.: US 2024/0409848 A1
(43) Pub. Date: Dec. 12, 2024

(54) ANTIMICROBIAL CLEANING COMPOSITIONS WITH HARD WATER TOLERANCE

(71) Applicant: ECOLAB USA INC., Saint Paul, MN (US)

(72) Inventors: Derrick Anderson, Saint Paul, MN (US); Junzhong Li, Saint Paul, MN (US); Rosalind Haas, Saint Paul, MN (US); Joshua Luedtke, Saint Paul, MN (US); Richard Staub, Saint Paul, MN (US)

(21) Appl. No.: 18/624,327

(22) Filed: Apr. 2, 2024

Related U.S. Application Data
(60) Provisional application No. 63/506,498, filed on Jun. 6, 2023.

(51) Int. Cl. C11D 3/48; A01N 37/02; A01N 37/36; A01N 41/02; A01N 41/04; A01P 1/00; C11D 1/14; C11D 1/22; C11D 1/66; C11D 1/72; C11D 1/83; C11D 3/20; C11D 3/37

(52) U.S. Cl. CPC C11D 3/48; A01N 37/02; A01N 37/36; A01N 41/02; A01N 41/04; A01P 1/00; C11D 1/83; C11D 3/2079; C11D 3/2086; C11D 3/3707; C11D 1/143; C11D 1/146; C11D 1/22; C11D 1/662; C11D 1/72

(57) ABSTRACT

Disclosed are hard water-resistant antimicrobial composition for use in cleaning surfaces, wherein the compositions comprise a carboxylic acid, an alkyl sulfate, and a sulfonate. Further disclosed are methods of using the same, wherein the antimicrobial compositions provides an at least a 3-log reduction of a microbial population within 10 minutes of contact with the surface. Beneficially, the compositions are stable in hard water, do not require a rinse step, and leave little or no residue on treated surfaces.

### CROSS-REFERENCE TO RELATED APPLICATIONS

This application claims priority under 35 U.S.C. § 119 to Provisional Application U.S. Ser. No. 63/506,498, filed Jun. 6, 2023, which is herein incorporated by reference in its entirety including without limitation, the tables, examples, and claims.

### TECHNICAL FIELD

The present disclosure relates to antimicrobial compositions and more particularly to stable, hard water-tolerant antimicrobial compositions comprising a sulfate, sulfonate, and at least one organic acid, namely a carboxylic acid.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Antimicrobial compositions have long been used to clean and sanitize surfaces in various settings, such as hospitals, food processing plants, and households. In recent years, there has been an increasing demand for environmentally friendly and effective antimicrobial compositions that comply with regulatory requirements. One such requirement is set forth by the U.S. Department of Transportation (DOT), which requires strict measures for transportation of products that contain strong acids owing to their corrosive hazard.

In the absence of strong acids, alkyl alcohol sulfates such as sodium lauryl sulfate, may be used to deliver antimicrobial performance. Alkyl alcohol sulfates have been shown to have antimicrobial efficacy under broader pH conditions, which is a significant advantage compared to other surfactants, such as linear alkyl benzene sulfonate, which have a strong pH-dependent efficacy. However, alkyl alcohol sulfates have poor stability in hard water.

There is therefore a need to address the problem of antimicrobial efficacy in hard water. Other compositions utilize nonionic surfactants and polymers as potential stabilizers for alkyl alcohol sulfates. However, it was found that these stabilizers caused significant streaking on hard surfaces, rendering them unsuitable for use in antimicrobial compositions.

There is additionally a need to develop antimicrobial compositions that maintain good antimicrobial efficacy under challenging use conditions, particularly in conditions involving hard water.

Other objects, embodiments and advantages of this disclosure will be apparent to one skilled in the art in view of the following disclosure, the drawings, and the appended claims.

### BRIEF SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

The disclosure provides for antimicrobial compositions comprising at least one carboxylic acid; an alkyl sulfate; and a sulfonate. In further embodiments the antimicrobial compositions comprise a carboxylic acid, an alkyl sulfate, and a sulfonate. In an embodiment, the carboxylic acid may comprise acetic acid, formic acid, butyric acid, benzoic acid, propionic acid, valeric acid, caproic acid, palmitic acid, stearic acid, oleic acid, or a combination thereof. In an embodiment, the compositions further comprise a second carboxylic acid. According to an embodiment, the second polycarboxylic acid may comprise citric acid, oxalic acid, succinic acid, adipic acid, malic acid, tartaric acid, or a combination thereof. In an embodiment, the composition may further comprise an additional acid, such as methane sulfonic acid, sulfuric acid, sodium hydrogen sulfate, phosphoric acid, phosphonic acid, nitric acid, sulfamic acid, hydrochloric acid, trichloroacetic acid, trifluoroacetic acid, toluene sulfonic acid, glutamic acid, or a combination thereof.

In an embodiment, the alkyl sulfate may comprise a C8-C18 alkyl sulfate. In an embodiment, the alkyl sulfate may comprise sodium lauryl sulfate (SLS), sodium laureth sulfate (SLES), ammonium lauryl sulfate (ALS), or a combination thereof. In an embodiment, the sulfonate may comprise an alpha olefin sulfonate, alkane sulfonate, alkyl sulfonate, sulfonated carboxylic acid ester, or a combination thereof. In another embodiment, the sulfonate may comprise an alpha olefin sulfonate, wherein the alpha olefin sulfonate is a C8-C22 alpha olefin sulfonate.

In an embodiment, the composition may further comprise an additional functional ingredient, which may comprise a nonionic surfactant, alkylpolyglucoside, additional anionic surfactant, cationic surfactant, or a combination thereof. In an embodiment, the composition may comprise from about 10 wt. % to about 25 wt. % of the alkyl sulfate; from about 20 wt. % to about 35 wt. % of the sulfonate; from about 40 wt. % to about 60 wt. % of the carboxylic acid; and from about 0.5 wt. % to about 10 wt. % of the polycarboxylic acid.

Also provided are methods of using the antimicrobial composition, the method comprising contacting an antimicrobial composition with a surface at a use concentration between about 0.2% to about 20% (active basis), and providing at least a 3-log reduction of a microbial population. In an embodiment, the composition may be diluted to form a use solution before the contacting. In an embodiment, the at least 3-log reduction occurs within 10 minutes of the contacting. In an embodiment, the contacting may be by wiping, dipping, immersing, or spraying. In an embodiment, the composition does not leave streaks on the surface. In an embodiment, the surface is a hard surface. In an embodiment, the contacting step is at an aqueous use temperature from about 20° C. to about 71° C. In a further embodiment, the method does not require a rinse step before the contacting, after the contacting, or a combination thereof.

It is a primary object, feature, and/or advantage of the present disclosure to improve on or overcome the deficiencies in the art.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### DETAILED DESCRIPTION

The present disclosure relates to antimicrobial cleaning compositions with hard water tolerance. The compositions compose acids, anionic surfactants and an alkane olefin sulfonate that provides hardwater tolerance. The compositions are efficacious against a broad spectrum of microorganisms including viruses and can be used for food contact surfaces without subsequent rinsing.

It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

As used herein, the term “and/or,” e.g., “X and/or Y” shall be understood to mean either “X and Y” or “X or Y” and shall be taken to provide explicit support for both meanings or for either meaning, e.g. A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

The methods and compositions of the present disclosure may comprise, consist essentially of, or consist of the components and ingredients of the present disclosure as well as other ingredients described herein. As used herein, “consisting essentially of” means that the compositions and methods may include additional steps, components or ingredients, but only if the additional steps, components or ingredients do not materially alter the basic and novel characteristics of the claimed compositions and methods.

Unless defined otherwise, all technical and scientific terms used above have the same meaning as commonly understood by one of ordinary skill in the art to which embodiments of the present disclosure pertain.

The term “about,” as used herein, refers to variation in the numerical quantity that can occur, for example, through typical measuring techniques and equipment, with respect to any quantifiable variable, including, but not limited to, concentration, mass, volume, time, molecular weight, temperature, pH, humidity, molar ratios, log count of bacteria or viruses, and the like. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts. It is also sometimes indicated by a percentage in parentheses, for example, “chemical (10%).”

As used herein, the term “alkyl” or “alkyl groups” refers to saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (e.g., isopropyl, tert-butyl, sec-butyl, isobutyl, etc.), and alkyl-substituted alkyl groups (e.g., alkyl-substituted cycloalkyl groups and cycloalkyl-substituted alkyl groups).

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and urcido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Example heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

Differentiation of antimicrobial “-cidal” or “-static” activity, the definitions which describe the degree of efficacy, and the official laboratory protocols for measuring this efficacy are considerations for understanding the relevance of antimicrobial agents and compositions. Antimicrobial compositions can affect two kinds of microbial cell damage. The first is a lethal, irreversible action resulting in complete microbial cell destruction or incapacitation. The second type of cell damage is reversible, such that if the organism is rendered free of the agent, it can again multiply. The former is termed microbiocidal and the later, microbiostatic. A sanitizer and a disinfectant are, by definition, agents which provide antimicrobial or microbiocidal activity. In contrast, a preservative is generally described as an inhibitor or microbiostatic composition. As referred to herein, antimicrobial compositions are further suitable for microbiocidal activity against viral pathogens, including for example, Norovirus and Murine Norovirus.

As used herein, the term “antimicrobial” refers to a compound or composition that reduces and/or inactivates a microbial population, including, but not limited to bacteria, viruses, fungi, and algae within about 10 minutes or less, about 8 minutes or less, about 5 minutes or less, about 3 minutes or less, about 2 minutes or less, about 1 minute or less, or about 30 seconds or less. Preferably, the term antimicrobial refers to a composition that provides at least about a 3-log, 3.5-log, 4-log, 4.5-log, or 5-log reduction of a microbial population in about 10 minutes or less, about 8 minutes or less, about 5 minutes or less, about 3 minutes or less, about 2 minutes or less, about 1 minute or less, or about 30 seconds or less.

As used herein, the term “cleaning” refers to a method used to facilitate or aid in soil removal, bleaching, microbial population reduction, rinsing, and any combination thereof.

The term “commercially acceptable cleaning performance” refers generally to the degree of cleanliness, extent of effort, or both that a typical consumer would expect to achieve or expend when using a cleaning product or cleaning system to address a typical soiling condition on a typical substrate. This degree of cleanliness may, depending on the particular cleaning product and particular substrate, correspond to a general absence of visible soils, or to some lesser degree of cleanliness. Cleanliness may be evaluated in a variety of ways depending on the particular product being used and the particular surface being cleaned, and normally may be determined using generally agreed industry standard tests or localized variations of such tests. In some embodiments, the methods providing virucidal efficacy also provide commercially acceptable cleaning performance while ensuring the formulations do not leave hazy, streaky, or tacky residues on treated surfaces.

As used herein, the term “disinfectant” refers to an agent that kills all vegetative cells including most recognized pathogenic microorganisms. In an embodiment, disinfectant capability can be evaluated using any suitable procedure, such as the EPA OCSPP 810.2200 Disinfectants for Use on Environmental Surfaces, Guide for Efficacy Testing (February 2018) and/or use dilution methods such as AOAC Use Dilution techniques, particularly 964.02 for Pseudomonas aeruginosa, 955.14 or Salmonella enterica, and 955.15 for Staphylococcus aureus. As used herein, the term “high level disinfection” or “high level disinfectant” refers to a compound or composition that kills substantially all organisms, except high levels of bacterial spores, and is affected with a chemical germicide cleared for marketing as a sterilant by the Food and Drug Administration. As used herein, the term “intermediate-level disinfection” or “intermediate level disinfectant” refers to a compound or composition that kills mycobacteria, most viruses, and bacteria with a chemical germicide registered as a tuberculocide by the Environmental Protection Agency (EPA). As used herein, the term “low-level disinfection” or “low level disinfectant” refers to a compound or composition that kills some viruses and bacteria with a chemical germicide registered as a hospital disinfectant by the EPA.

According to various embodiments of the methods and compositions described herein, the AOAC Method 960.09 methodology was used to demonstrate bactericidal performance with a 5-log reduction requirement.

As used herein, the phrase “food processing surface” refers to a surface of a tool, a machine, equipment, a structure, a building, or the like that is employed as part of a food processing, preparation, or storage activity. Examples of food processing surfaces include surfaces of food processing or preparation equipment (e.g., slicing, canning, or transport equipment, including flumes), of food processing wares (e.g., utensils, dishware, wash ware, and bar glasses), and of floors, walls, or fixtures of structures in which food processing occurs. Food processing surfaces are found and employed in food anti-spoilage air circulation systems, aseptic packaging sanitizing, food refrigeration and cooler cleaners and sanitizers, ware washing sanitizing, blancher cleaning and sanitizing, food packaging materials, cutting board additives, third-sink sanitizing, beverage chillers and warmers, meat chilling or scalding waters, autodish sanitizers, sanitizing gels, cooling towers, food processing antimicrobial garment sprays, and non-to-low-aqueous food preparation lubricants, oils, and rinse additives.

As used herein, the term “free” refers to compositions completely lacking the component or having such a small amount of the component that the component does not affect the performance of the composition.

The term “generally” encompasses both “about” and “substantially.”

The term “hard surface” refers to a solid, substantially non-flexible surface such as a countertop, tile, floor, wall, panel, window, plumbing fixture, kitchen and bathroom furniture, appliance, engine, circuit board, dish, mirror, window, monitor, touch screen, and thermostat. Hard surfaces are not limited by the material; for example, a hard surface can be glass, metal, tile, vinyl, linoleum, composite, wood, plastic, etc. Hard surfaces may include for example, health care surfaces and food processing surfaces.

As used herein, the phrase “health care surface” refers to a surface of an instrument, a device, a cart, a cage, furniture, a structure, a building, or the like that is employed as part of a health care activity. Examples of health care surfaces include surfaces of medical or dental instruments, of medical or dental devices, of electronic apparatus employed for monitoring patient health, and of floors, walls, or fixtures of structures in which health care occurs. Health care surfaces are found in hospital, surgical, infirmity, birthing, mortuary, and clinical diagnosis rooms. These surfaces can be those typified as “hard surfaces” (such as walls, floors, bed-pans, etc.), or fabric surfaces, e.g., knit, woven, and non-woven surfaces (such as surgical garments, draperies, bed linens, bandages, etc.), or patient-care equipment (such as respirators, diagnostic equipment, shunts, body scopes, wheel chairs, beds, etc.), or surgical and diagnostic equipment. Health care surfaces include articles and surfaces employed in animal health care.

As used herein, the term “instrument” refers to the various medical or dental instruments or devices that can benefit from cleaning with a composition as described herein.

As used herein, the term “microorganism” refers to any noncellular or unicellular (including colonial) organism. Microorganisms include all prokaryotes. Microorganisms include bacteria (including cyanobacteria), spores, lichens, fungi, protozoa, virinos, viroids, viruses, phages, and some algae. As used herein, the term “microbe” is synonymous with microorganism.

The term “norovirus” is meant to refer to the human norovirus (referred to simply as norovirus) which is in the family Caliciviridae, which is the leading cause of acute nonbacterial gastroenteritis. There are various surrogates commonly used for norovirus as to date, human norovirus cannot be grown in cell culture. Norovirus has a low infectious dose (10 to 100 virus particles), and environmental contamination prolongs outbreaks. Surfaces, serving dishes or containers, utensils, and food handled by ill persons who are not practicing adequate personal hygiene before preparing food may also contribute to illness. Feline calicivirus (FCV), from the genus Vesivirus, can be propagated in cell culture, it has been extensively studied as a surrogate for human norovirus in environmental survival and inactivation studies. However, FCV is transmitted by the respiratory route and is inactivated at a relatively low pH, and hence, it may not predict human norovirus environmental stability or inactivation. Mouse norovirus 1 (MNV-1) has been propagated in cell culture and causes a lethal infection in mice that presents as hepatitis, pneumonia, or inflammation of the nervous system and is therefore very different from the clinical presentation of the human norovirus; however, MNV-1 is shed in mouse feces and is commonly transmitted by the fecal-oral route. The genetic relatedness of MNV-1 to norovirus combined with its ability to survive under gastric pH levels (minimal loss of infectivity at pH 2) makes this virus a relevant surrogate for studying environmental survival of norovirus. The MNV-1 is able to survive low pH and is superior in acid tolerance in comparison to FCV.

As used herein the term “polymer” refers to a molecular complex comprised of a more than ten monomeric units and generally includes, but is not limited to, homopolymers, copolymers, such as for example, block, graft, random and alternating copolymers, terpolymers, and higher “x” mers, further including their analogs, derivatives, combinations, and blends thereof. Furthermore, unless otherwise specifically limited, the term “polymer” shall include all possible isomeric configurations of the molecule, including, but are not limited to isotactic, syndiotactic and random symmetries, and combinations thereof. Furthermore, unless otherwise specifically limited, the term “polymer” shall include all possible geometrical configurations of the molecule.

As used herein, the term “sanitizer” refers to an agent that reduces the number of bacterial contaminants to safe levels as judged by public health requirements. In an embodiment, sanitizers for use in this disclosure will provide at least a 99.999% reduction (5-log order reduction). These reductions can be evaluated using a procedure set out in Germicidal and Detergent Sanitizing Action of Disinfectants, Official Methods of Analysis of the Association of Official Analytical Chemists, paragraph 960.09 and applicable sections, 15th Edition, 1990 (EPA Guideline 91-2). According to this reference a sanitizer should provide a 99.999% reduction (5-log order reduction) within 30 seconds at room temperature, 25±2° C., against several test organisms.

As used herein, the term “soil” or “stain” refers to any soil, including, but not limited to, non-polar oily and/or hydrophobic substances which may or may not contain particulate matter such as industrial soils, mineral clays, sand, natural mineral matter, carbon black, graphite, kaolin, environmental dust, and/or food-based soils such as blood, proteinaceous soils, starchy soils, fatty soils, cellulosic soils, etc.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, sub combinations, or the like that would be obvious to those skilled in the art.

The term “substantially” refers to a great or significant extent. “Substantially” can thus refer to a plurality, majority, and/or a supermajority of said quantifiable variable, given proper context.

As used herein, the term “substantially free” refers to compositions completely lacking the component or having such a small amount of the component that the component does not affect the performance of the composition. The component may be present as an impurity or as a contaminant and shall be less than 0.5 wt. %. In another embodiment, the amount of the component is less than 0.1 wt. % and in yet another embodiment, the amount of component is less than 0.01 wt. %.

The term “surfactant” or “surface active agent” refers to an organic chemical that when added to a liquid changes the properties of that liquid at a surface.

As used herein, the term “virucidal” refers to an agent that reduces the number of viruses on a surface or substrate. In an embodiment, virucidal compositions will provide at least a 3-log order reduction, or preferably a 5-log order reduction, or more preferably a complete inactivation of viruses. These reductions can be evaluated using a procedure set out in ASTM E1053 Standard Test Method for Efficacy of Virucidal Agents Intended for Inanimate Environmental Surfaces; US standards are set forth in EPA 810.2200; EP standards are set forth in EN 14476, each of which are herein incorporated by reference in its entirety. The outlined log reductions can be achieved over various periods of time (which can vary according to contact time requirements set forth in various jurisdictions), including for example less than about 60 minutes, less than about 30 minutes, less than about 5 minutes, less than 1 minute, less than about 30 seconds, or even less than about 15 seconds. According to this reference a virucidal composition should provide a 99.9% reduction (3-log order reduction) for virucidal activity.

The term “virus,” as used herein refers to a type of microorganism that can include both pathogenic and non-pathogenic viruses. Pathogenic viruses can be classified into two general types with respect to the viral structure: enveloped viruses and non-enveloped viruses. Some well-known enveloped viruses include herpes virus, influenza virus; paramyxovirus, respiratory syncytial virus, corona virus, HIV, hepatitis B virus, hepatitis C virus and SARS-CoV virus. Non-enveloped viruses, sometimes referred to as “naked” viruses, include the families Picornaviridae, Reoviridae, Caliciviridae, Adenoviridae and Parvoviridae. Members of these families include rhinovirus, poliovirus, adenovirus, hepatitis A virus, norovirus, papillomavirus, and rotavirus. It is known in the art that “enveloped” viruses are relatively sensitive and, thus, can be inactivated by commonly used disinfectants. In contrast, non-enveloped viruses are substantially more resistant to conventional disinfectants and are significantly more environmentally stable than enveloped viruses.

As used herein, the term “waters” includes various water sources. Water temperatures can range from about 40° F.-160° F., about 60° F.-140° F., or about 70° F.-140° F.

The term “water soluble” refers to a compound that can be dissolved in water at a concentration of more than 1 wt. %. The terms “sparingly soluble” or “sparingly water soluble” refer to a compound that can be dissolved in water only to a concentration of 0.1 to 1.0 wt. %. The term “water insoluble” refers to a compound that can be dissolved in water only to a concentration of less than 0.1 wt. %.

The term “weight percent,” “wt. %,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt. %,” etc.

### Compositions

Example ranges of the antimicrobial compositions are presented in Tables 1A and 1B, wherein the compositions are shown as liquid concentrate formulations on a weight percentage basis. While the components may have a percent actives of 100%, it is noted that Tables 1A, 1B and 1C do not recite the percent actives of the components, but rather, recites the total weight percentage of the raw materials (i.e., active concentration plus inert ingredients).

TABLE 1A

| | First | Second | Third |
|---|---|---|---|
| | Example | Example | Example |
| Material | Range wt. % | Range wt. % | Range wt. % |
| Sulfate Surfactant | 5-40 | 10-35 | 15-35 |
| Sulfonate Surfactant | 5-50 | 10-35 | 15-25 |
| First Carboxylic Acid | 1-60 | 25-60 | 30-60 |
| Second Carboxylic Acid | 0-60 | 0-40 | 0-8 |
| Optional Additional | 0.01-90 | 0.01-30 | 0.01-25 |
| Functional Ingredients | | | |

TABLE 1B

| | First | Second | Third |
|---|---|---|---|
| | Example | Example | Example |
| Material | Range wt. % | Range wt. % | Range wt. % |
| Alkyl Sulfate Surfactant | 10-35 | 15-35 | 25-35 |
| Alkane Olefin Sulfonate | 15-35 | 15-30 | 15-25 |
| Surfactant | | | |
| First Carboxylic Acid | 30-60 | 35-55 | 35-50 |
| Second Carboxylic Acid | 0-10 | 0-8 | 1-5 |
| Optional Additional | 0-50 | 0-30 | 0-25 |
| Functional Ingredients | | | |

TABLE 1C

| | First | Second | Third |
|---|---|---|---|
| | Example | Example | Example |
| Material | Range wt. % | Range wt. % | Range wt. % |
| Alkyl Sulfate Surfactant | 10-35 | 15-35 | 25-35 |
| Alkane Olefin Sulfonate | 15-35 | 15-30 | 15-25 |
| Surfactant | | | |
| First Carboxylic Acid | 30-60 | 35-55 | 35-50 |
| (hydroxy acid and/or | | | |
| polycarboxylic acid) | | | |
| Second Carboxylic Acid | 1-10 | 1-8 | 1-5 |
| (fatty acid) | | | |
| Optional Additional | 0-50 | 0-30 | 0-25 |
| Functional Ingredients | | | |

The antimicrobial compositions may include concentrate compositions which can be diluted to form use compositions or ready to use (RTU) compositions. Beneficially, the compositions overcome a limitation of the prior art in that dilutable concentrates can be provided. In general, a concentrate refers to a composition that is intended to be diluted with water to provide a use solution that contacts an object to provide the desired cleaning, antimicrobial efficacy, or the like. The antimicrobial compositions that contacts the articles can be referred to as a concentrate or a use composition (or use solution) dependent upon the formulation employed in the methods described herein. It should be understood that the concentration of the components in the composition will vary depending on whether the composition is provided as a concentrate or as a use solution. One skilled in the art can adjust the percent (%) by weight of the compositions to arrive at a composition having a different dilution rate, which is within the scope of the disclosed compositions. Beneficially, within the ranges of actives, the compositions can be formulated to include a nearly or completely waterless liquid or solid composition.

A use solution may be prepared from the concentrate by diluting the liquid concentrate with water at a dilution ratio that provides a use solution having desired antimicrobial properties. The water that is used to dilute the concentrate to form the use composition can be referred to as water of dilution or a diluent and can vary from one location to another. The typical dilution factor is between approximately 1 and approximately 10,00. In an embodiment, the liquid concentrate is diluted at a ratio of between about 1:10 and about 1:10,00 liquid concentrate to water, between about 1:10 and about 1:1,000 liquid concentrate to water, or between about 1:10 and about 1:510 liquid concentrate to water.

In an embodiment, a diluted use solution is made from about a 0.25% to about a 5% by weight dilution of the liquid concentrate composition. In another embodiment, a concentrate can be diluted at a concentration from about 0.25% to about 2.0%, from about 0.50% to about 1.5%, or from about 0.5% to about 1.0% while providing sanitizing efficacy.

In another embodiment, a concentrate can be diluted at a concentration from about 0.25% to about 5.0%, from about 0.5% to about 5.0%, or from about 1.0% to about 5.0% while providing disinfecting efficacy.

The liquid compositions can be provided in various forms well appreciated by those skilled in the art. The compositions can also be manufactured to include a saturated antimicrobial wipe, such as a paper or cloth substrate having the liquid compositions saturated thereon. In embodiments, the liquid compositions are provided as liquid concentrates. In other embodiments, the liquid compositions are provided as ready to use liquids, such as for example a ready to use spray. Beneficially according to various embodiments the spray compositions do not require potable water rinse at use conditions due to efficacy of the compositions at use ppm concentration ranges.

### Organic Acids

The antimicrobial compositions comprise at least one acid and preferably at least two organic acids, and particularly at least two carboxylic acids. Preferably, the organic acid(s) provide a pH of less than or equal to about 5 in a use solution of the composition and a concentrate product pH less than about 2, or from about 1-2. As used herein, organic acid refers to a compound containing one or more carboxyl groups (—COOH). The carboxyl group is a functional group consisting of a carbon atom with a double bond to an oxygen atom and a single bond to a hydroxyl group (—OH). Organic acids are typically weak acids, meaning they do not completely dissociate in solution and generally have a pH less than 7. In an embodiment, the compositions include one or more organic acids having a pKa greater than about 2.5 to beneficially provide use solutions having a pH less than about 5, less than about 4, or preferably less than about 3.

Carboxylic acids are a class of organic compounds that contain at least one carboxyl group (—COOH) in their molecular structure. The carboxyl group consists of a carbonyl group (C═O) and a hydroxyl group (—OH) attached to the same carbon atom. The term “carboxylic acid” includes hydroxy acids, polycarboxylic acids, and fatty acids. Examples of suitable carboxylic acids include, without limitation, acetic acid, citric acid, formic acid, butyric acid, benzoic acid, propionic acid, octanoic acid, nonanoic acid, decanoic acid, palmitic acid, stearic acid, tartaric acid, lactic acid, oleic acid, palmitic acid, citric acid, malic acid, succinic acid, oxalic acid or a combination thereof. In a preferred embodiment, the compositions comprise a first carboxylic acid and a second carboxylic acid.

Hydroxy acids are a sub-class of carboxylic acids that consist of a carboxylic acid with a hydroxyl group on the alkyl chain. Alpha hydroxy carboxylic acids consist of a carboxylic acid with a hydroxyl group substituent on the adjacent alpha carbon. Some examples of suitable alpha-hydroxy acids include, without limitation, glycolic acid, lactic acid, malic acid, mandelic acid, citric acid, tartaric acid, or a combination thereof.

Polycarboxylic acids are a type of carboxylic acid that contain multiple carboxylic acid functional groups. Some common examples of suitable polycarboxylic acids include citric acid, oxalic acid, succinic acid, adipic acid, malic acid, tartaric acid, glutaric acid or a combination thereof.

Fatty acids are long-chain carboxylic acids that are commonly found in various natural fats and oils. Suitable fatty acids include, for example, heptanoic acid, hexanoic acid, caproic acid, caprylic acid, capric acid, palmitic acid, stearic acid, oleic acid, nonanoic acid, linoleic acid, arachidonic acid, or a combination thereof.

An additional type of organic acid includes a phenolic acid which can optionally be included as an additional acid in the compositions. In other embodiments the compositions are free of phenolic acids. Phenolic acids are a class of organic compounds that contain a phenolic ring and a carboxylic acid group. Some examples of suitable phenolic acids include, without limitation, caffeic acid, ferulic acid, gallic acid, protocatechuic acid, vanillic acid, or a combination thereof. An additional type of organic acid includes a sulfonic acid which can optionally be included as an additional acid in the compositions. In other embodiments the compositions are free of sulfonic acids. Sulfonic acids are a class of organic compounds that contain a sulfonic acid group (—SO₃H) attached to a carbon atom. Some examples of sulfonic acids include, without limitation, benzenesulfonic acid, toluene sulfonic acid, methane sulfonic acid, sulfamic acid, or a combination thereof.

In sum, examples of suitable acids include, without limitation, acetic acid, citric acid, formic acid, butyric acid, benzoic acid, propionic acid, tartaric acid, lactic acid, oleic acid, palmitic acid, glycolic acid, lactic acid, malic acid, mandelic acid, citric acid, tartaric acid, palmitic acid, stearic acid, oleic acid, linoleic acid, arachidonic acid, caffeic acid, ferulic acid, gallic acid, protocatechuic acid, vanillic acid, benzenesulfonic acid, toluene sulfonic acid, methane sulfonic acid, sulfamic acid, citric acid, oxalic acid, succinic acid, adipic acid, malic acid, tartaric acid, or a combination thereof.

In embodiments, the compositions include from about 1 wt. % to about 80 wt. % of one or more organic acids, or from about 1 wt. % to about 60 wt. % of one or more organic acids. In addition, without being limited according to the disclosure, all ranges recited are inclusive of the numbers defining the range and include each integer within the defined range.

In a preferred embodiment, the compositions comprise a first organic acid and a second organic acid. In a further preferred embodiment, the compositions comprise a carboxylic acid and a polycarboxylic acid. In a still further preferred embodiment, the carboxylic acid is lactic acid and the polycarboxylic acid is citric acid.

In an alternative preferred embodiment, the compositions comprise a first organic acid and a second organic acid, preferably a first carboxylic acid and a second carboxylic acid. In a still further embodiment, the first carboxylic acid comprises a hydroxy acid and/or polycarboxylic acid, and the second carboxylic acid comprises a fatty acid. In a still further embodiment, the first carboxylic acid comprises citric acid and/or lactic acid, and the second carboxylic acid comprises nonanoic acid.

In embodiments, the compositions include from about 1 wt. % to about 60 wt. %, from about 25 wt. % to about 60 wt. %, or from about 30 wt. % to about 60 wt. % of the first carboxylic acid and from about 0 wt. % to about 60 wt. %, from about 0 wt. % to about 40 wt. %, or from about 0 wt. % to about 8 wt. % of the second carboxylic acid, including any combinations thereof with a preference for the second carboxylic acid being greater than 0 wt. % (i.e. the second carboxylic acid is present in the composition).

In embodiments, the compositions include from about 30 wt. % to about 60 wt. %, from about 35 wt. % to about 55 wt. %, or from about 35 wt. % to about 50 wt. % of the first carboxylic acid and from about 0 wt. % to about 10 wt. %, from about 0 wt. % to about 8 wt. %, or from about 1 wt. % to about 5 wt. % of the second carboxylic acid, including any combinations thereof with a preference for the second carboxylic acid being greater than 0 wt. % (i.e. the second carboxylic acid is present in the composition).

### Optional Additional Acids

The compositions optionally include one or more strong acids which substantially dissociate an aqueous solution. The strong acid may include any strong acid, whether organic or inorganic.

Examples of strong acids suitable for use include methane sulfonic acid, sulfuric acid, sodium hydrogen sulfate, phosphoric acid, phosphonic acid, nitric acid, sulfamic acid, hydrochloric acid, trichloroacetic acid, trifluoroacetic acid, toluene sulfonic acid, glutamic acid, and the like; alkane sulfonic acid, such as methane sulfonic acid, sodium bisulfate, ethane sulfonic acid, linear alkyl benzene sulfonic acid, xylene sulfonic acid, cumene sulfonic acid and the like.

In certain embodiments, the compositions include from about 0 wt. % to about 20 wt. % of a strong acid, from about 0.1 wt. % to about 20 wt. % of a strong acid, from about 5 wt. % to about 20 wt. % of a strong acid, or from about 5 wt. % to about 15 wt. % of a strong acid. In addition, without being limited according to the disclosure, all ranges recited are inclusive of the numbers defining the range and include each integer within the defined range.

In preferred embodiments the compositions are free of strong acids.

### Anionic Sulfate Surfactants

In preferred embodiments, the compositions comprise at least one anionic sulfate surfactant, preferably an alkyl sulfate surfactant. Sulfate surfactants are a type of anionic surfactant derived from sulfuric acid and an alkyl group. Frequently the alkyl group is a hydrocarbon containing 8-18 carbon atoms. Examples of suitable anionic sulfate surfactants include alkyl ether sulfates, alkyl sulfates, the linear and branched primary and secondary alkyl sulfates, alkyl ethoxysulfates, fatty oleyl glycerol sulfates, alkyl phenol ethylene oxide ether sulfates, the C₅-C₁₇ acyl-N—(C₁-C₄ alkyl) and —N—(C₁-C₂ hydroxyalkyl) glucamine sulfates, and sulfates of alkylpolysaccharides such as the sulfates of alkylpolyglucoside, and the like. Also included are the alkyl sulfates, alkyl poly(ethyleneoxy) ether sulfates and aromatic poly(ethyleneoxy) sulfates such as the sulfates or condensation products of ethylene oxide and nonyl phenol (usually having 1 to 6 oxyethylene groups per molecule).

Particularly preferred types of sulfate surfactants comprise alkyl sulfates, in particular C₈-C₁₈ alkyl sulfates. Still further, preferred sulfate surfactants comprise sodium lauryl sulfate (SLS), sodium laureth sulfate (SLES), ammonium lauryl sulfate (ALS), or a combination thereof.

In certain embodiments, the compositions include from about 5 wt. % to about 50 wt. % of a sulfate surfactant, from about 5 wt. % to about 40 wt. % of a sulfate surfactant, from about 10 wt. % to about 35 wt. % of a sulfate surfactant, from about 15 wt. % to about 35 wt. % of a sulfate surfactant, from about 10 wt. % to about 30 wt. % of a sulfate surfactant, or from about 15 wt. % to about 20 wt. % of a sulfate surfactant. In an embodiment the compositions preferably include from about 5 wt. % to about 20 wt. % of a sulfate surfactant. In an embodiment the compositions preferably include at least about 40 wt. % of the combined alkyl sulfate and the sulfonate anionic surfactant, or preferably from about 40 wt. % to about 90 wt. %, about 40 wt. % to about 70 wt. %, about 40 wt. % to about 65 wt. %, or about 40 wt. % to about 60 wt. % of the combined alkyl sulfate and the sulfonate anionic surfactant. In addition, without being limited according to the disclosure, all ranges recited are inclusive of the numbers defining the range and include each integer within the defined range.

### Anionic Sulfonate Surfactant

In an embodiment, the compositions include at least one sulfonate surfactant. Sulfonate surfactants are generally derived from a sulfonic acid group and typically contain a hydrophobic alkyl or aryl group. Examples of suitable surfactants include, but are not limited to, an alpha olefin sulfonate (also referred to as alkyl olefin sulfonate, or AOS), alkane sulfonate, alkyl sulfonate, sulfonated carboxylic acid ester, alkyl polyglucoside, phosphate ester, or a combination thereof.

In an embodiment, an anionic sulfonate surfactant is an alkyl sulfonate, including linear and branched primary and secondary alkyl sulfonates, and the aromatic sulfonates with or without substituents. In a preferred embodiment the anionic sulfonate surfactant is an alpha olefin sulfonate or a salt thereof. Alpha olefin sulfonates are available as aqueous solutions, powders or as a solid anhydrous product. Preferred anionic sulfonates include C8-C22 alpha olefin sulfonates, or C8-C16 alpha olefin sulfonates.

In still further embodiment, anionic sulfonate surfactant comprises an alkyl benzene sulfonate (ABS), linear alkyl sulfonate (LAS), and sodium lauryl ether sulfate (SLES), or a combination thereof. In preferred embodiments the compositions do not include and are free of an alkyl benzene sulfonate (ABS), linear alkyl sulfonate (LAS), and sodium lauryl ether sulfate (SLES), or a combination thereof.

In certain embodiments, the compositions include from about 5 wt. % to about 50 wt. % of a sulfonate surfactant, from about 10 wt. % to about 35 wt. % of a sulfonate surfactant, from about 15 wt. % to about 35 wt. % of a sulfonate surfactant, from about 10 wt. % to about 30 wt. % of a sulfonate surfactant, from about 15 wt. % to about 30 wt. % of a sulfonate surfactant, from about 15 wt. % to about 25 wt. % of a sulfonate surfactant, or from about 15 wt. % to about 20 wt. % of a sulfonate surfactant. In an embodiment the compositions preferably include from about 15 wt. % to about 25 wt. % of a sulfonate surfactant. In an embodiment the compositions preferably include at least about 40 wt. % of the combined alkyl sulfate and the sulfonate anionic surfactant, or preferably from about 40 wt. % to about 90 wt. %, about 40 wt. % to about 70 wt. %, about 40 wt. % to about 65 wt. %, or about 40 wt. % to about 60 wt. % of the combined alkyl sulfate and the sulfonate anionic surfactant.

In addition, without being limited according to the disclosure, all ranges recited are inclusive of the numbers defining the range and include each integer within the defined range.

### Additional Surfactants

In some embodiments, the compositions optionally comprise one or more additional surfactants. The additional surfactant can comprise an anionic surfactant, nonionic surfactant, amphoteric surfactant, zwitterionic surfactant, or combinations.

In an embodiment, the compositions include from about 1 wt. % to about 40 wt. %, from about 1 wt. % to about 30 wt. %, from about 2 wt. % to about 30 wt. %, from about 2 wt. % to about 20 wt. %, or from about 2 wt. % to about 10 wt. % of the additional surfactant(s). In addition, without being limited according to the disclosure, all ranges recited are inclusive of the numbers defining the range and include each integer within the defined range.

In an embodiment, the compositions include at least about 40 wt. %, at least about 45 wt. %, at least about 50 wt. %, at least about 55 wt. %, or at least about 60 wt. % of the total surfactants. In addition, without being limited according to the disclosure, all ranges recited are inclusive of the numbers defining the range and include each integer within the defined range.

### Nonionic Surfactants

Nonionic surfactants are generally characterized by the presence of an organic hydrophobic group and an organic hydrophilic group and are typically produced by the condensation of an organic aliphatic, alkyl aromatic or polyoxyalkylene hydrophobic compound with a hydrophilic alkaline oxide moiety which in common practice is ethylene oxide or a polyhydration product thereof, polyethylene glycol. Practically any hydrophobic compound having a hydroxyl, carboxyl, amino, or amido group with a reactive hydrogen atom can be condensed with ethylene oxide, or its polyhydration adducts, or its mixtures with alkoxylenes such as propylene oxide to form a nonionic surface-active agent. The length of the hydrophilic polyoxyalkylene moiety which is condensed with any particular hydrophobic compound can be readily adjusted to yield a water dispersible or water-soluble compound having the desired degree of balance between hydrophilic and hydrophobic properties.

Useful nonionic surfactants include:

1. Block polyoxypropylene-polyoxyethylene polymeric compounds based upon propylene glycol, ethylene glycol, glycerol, trimethylolpropane, and ethylenediamine as the initiator reactive hydrogen compound. Examples of polymeric compounds made from a sequential propoxylation and ethoxylation of initiator are commercially available under the trade names Pluronic® and Tetronic® manufactured by BASF Corp.

Pluronic® compounds are difunctional (two reactive hydrogens) compounds formed by condensing ethylene oxide with a hydrophobic base formed by the addition of propylene oxide to the two hydroxyl groups of propylene glycol. This hydrophobic portion of the molecule weighs from 1,000 to 4,000. Ethylene oxide is then added to sandwich this hydrophobe between hydrophilic groups, controlled by length to constitute from about 10% by weight to about 80% by weight of the final molecule. An example includes Pluronic 17R4.

Tetronic® compounds are tetra-functional block copolymers derived from the sequential addition of propylene oxide and ethylene oxide to ethylenediamine. The molecular weight of the propylene oxide hydrotype ranges from 500 to 7,000; and the hydrophile, ethylene oxide, is added to constitute from 10% by weight to 80% by weight of the molecule.

2. Condensation products of one mole of alkyl phenol wherein the alkyl chain, of straight chain or branched chain configuration, or of single or dual alkyl constituent, contains from 8 to 18 carbon atoms with from 3 to 50 moles of ethylene oxide. The alkyl group can, for example, be represented by diisobutylene, di-amyl, polymerized propylene, iso-octyl, nonyl, and di-nonyl. These surfactants can be polyethylene, polypropylene, and polybutylene oxide condensates of alkyl phenols. Examples of commercial compounds of this chemistry are available on the market under the trade names Igepal® manufactured by Rhone-Poulenc and Triton® manufactured by Union Carbide.

3. Condensation products of one mole of a saturated or unsaturated, straight or branched chain alcohol having from 6 to 24 carbon atoms with from 3 to 50 moles of ethylene oxide. The alcohol moiety can consist of mixtures of alcohols in the above delineated carbon range, or it can consist of an alcohol having a specific number of carbon atoms within this range. Examples of like commercial surfactant are available under the trade names Neodol® manufactured by Shell Chemical Co. and Alfonic® manufactured by Vista Chemical Co.

4. Condensation products of one mole of saturated or unsaturated, straight or branched chain carboxylic acid having from 8 to 18 carbon atoms with from 6 to 50 moles of ethylene oxide. The acid moiety can consist of mixtures of acids in the above defined carbon atoms range, or it can consist of an acid having a specific number of carbon atoms within the range. Examples of commercial compounds of this chemistry are available on the market under the trade names Nopalcol® manufactured by Henkel Corporation and Lipopeg® manufactured by Lipo Chemicals, Inc.

In addition to ethoxylated carboxylic acids, commonly called polyethylene glycol esters, other alkanoic acid esters formed by reaction with glycerides, glycerin, and polyhydric (saccharide or sorbitan/sorbitol) alcohols can be used. All of these ester moieties have one or more reactive hydrogen sites on their molecule which can undergo further acylation or ethylene oxide (alkoxide) addition to control the hydrophilicity of these substances. Care must be exercised when adding these fatty esters or acylated carbohydrates to compositions containing amylase and/or lipase enzymes because of potential incompatibility.

Examples of nonionic low foaming surfactants include:

5. Compounds from (1) which are modified, essentially reversed, by adding ethylene oxide to ethylene glycol to provide a hydrophile of designated molecular weight; and, then adding propylene oxide to obtain hydrophobic blocks on the outside (ends) of the molecule. The hydrophobic portion of the molecule weighs from 1,000 to 3,100 with the central hydrophile including 10% by weight to 80% by weight of the final molecule. These reverse Pluronics® are manufactured by BASF Corporation under the trade name Pluronic® R surfactants.

Likewise, the Tetronic® R surfactants are produced by BASF Corporation by the sequential addition of ethylene oxide and propylene oxide to ethylenediamine. The hydrophobic portion of the molecule weighs from 2,100 to 6,700 with the central hydrophile including 10% by weight to 80% by weight of the final molecule.

6. Compounds from groups (1), (2), (3) and (4) which are modified by “capping” or “end blocking” the terminal hydroxy group or groups (of multi-functional moieties) to reduce foaming by reaction with a small hydrophobic molecule such as propylene oxide, butylene oxide, benzyl chloride; and, short chain fatty acids, alcohols or alkyl halides containing from 1 to 5 carbon atoms; and mixtures thereof. Also included are reactants such as thionyl chloride which convert terminal hydroxy groups to a chloride group. Such modifications to the terminal hydroxy group may lead to all-block, block-heteric, heteric-block or all-heteric nonionics.

Additional examples of effective low foaming nonionics include:

7. The alkylphenoxypolyethoxyalkanols of U.S. Pat. No. 2,903,486 issued Sep. 8, 1959, to Brown et al. and represented by the formula

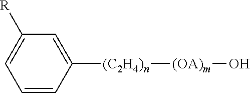

in which R is an alkyl group of 8 to 9 carbon atoms, A is an alkylene chain of 3 to 4 carbon atoms, n is an integer of 7 to 16, and m is an integer of 1 to 10.

The polyalkylene glycol condensates of U.S. Pat. No. 3,048,548 issued Aug. 7, 1962, to Martin et al. having alternating hydrophilic oxyethylene chains and hydrophobic oxypropylene chains where the weight of the terminal hydrophobic chains, the weight of the middle hydrophobic unit and the weight of the linking hydrophilic units each represent about one-third of the condensate.

The defoaming nonionic surfactants disclosed in U.S. Pat. No. 3,382,178 issued May 7, 1968 to Lissant et al. having the general formula Z[(OR)ₙOH]_{z} wherein Z is alkoxylatable material, R is a radical derived from an alkaline oxide which can be ethylene and propylene and n is an integer from, for example, 10 to 2,000 or more and z is an integer determined by the number of reactive oxyalkylatable groups.

The conjugated polyoxyalkylene compounds described in U.S. Pat. No. 2,677,700, issued May 4, 1954 to Jackson et al. corresponding to the formula Y(C₃H₆O)ₙ(C2H₄O)ₘH wherein Y is the residue of organic compound having from 1 to 6 carbon atoms and one reactive hydrogen atom, n has an average value of at least 6.4, as determined by hydroxyl number and m has a value such that the oxyethylene portion constitutes 10% to 90% by weight of the molecule.

The conjugated polyoxyalkylene compounds described in U.S. Pat. No. 2,674,619, issued Apr. 6, 1954 to Lundsted et al. having the formula Y [(C₃H₆Oₙ(C₂H₄O)ₘH]ₓ wherein Y is the residue of an organic compound having from 2 to 6 carbon atoms and containing x reactive hydrogen atoms in which x has a value of at least 2, n has a value such that the molecular weight of the polyoxypropylene hydrophobic base is at least 900 and m has value such that the oxyethylene content of the molecule is from 10% to 90% by weight. Compounds falling within the scope of the definition for Y include, for example, propylene glycol, glycerine, pentaerythritol, trimethylolpropane, ethylenediamine and the like. The oxypropylene chains optionally, but advantageously, contain small amounts of ethylene oxide and the oxyethylene chains also optionally, but advantageously, contain small amounts of propylene oxide.

Additional useful conjugated polyoxyalkylene surface-active agents correspond to the formula: P[(C₃H₆O)ₙ(C₂H₄O)ₘH]ₓ wherein P is the residue of an organic compound having from 8 to 18 carbon atoms and containing x reactive hydrogen atoms in which x has a value of 1 or 2, n has a value such that the molecular weight of the polyoxyethylene portion is at least 44 and m has a value such that the oxypropylene content of the molecule is from 10% to 90% by weight. In either case the oxypropylene chains may contain optionally, but advantageously, small amounts of ethylene oxide and the oxyethylene chains may contain also optionally, but advantageously, small amounts of propylene oxide.

8. Polyhydroxy fatty acid amide surfactants suitable for use in the present compositions include those having the structural formula R²CONR¹Z in which: R¹ is H, C₁-C₄ hydrocarbyl, 2-hydroxy ethyl, 2-hydroxy propyl, ethoxy, propoxy group, or a mixture thereof; R is a C₅-C₃ 1 hydrocarbyl, which can be straight-chain; and Z is a polyhydroxyhydrocarbyl having a linear hydrocarbyl chain with at least 3 hydroxyls directly connected to the chain, or an alkoxylated derivative (preferably ethoxylated or propoxylated) thereof. Z can be derived from a reducing sugar in a reductive amination reaction; such as a glycityl moiety.

9. The alkyl ethoxylate condensation products of aliphatic alcohols with from 0 to 25 moles of ethylene oxide are suitable for use in the present compositions. The alkyl chain of the aliphatic alcohol can either be straight or branched, primary or secondary, and generally contains from 6 to 22 carbon atoms.

10. The ethoxylated C₆-C₁₈ fatty alcohols and C₆-C₁₈ mixed ethoxylated and propoxylated fatty alcohols are suitable surfactants for use in the present compositions, particularly those that are water soluble. Suitable ethoxylated fatty alcohols include the C₁₀-C₁₈ ethoxylated fatty alcohols with a degree of ethoxylation of from 3 to 50.

11. Suitable nonionic alkylpolysaccharide surfactants, particularly for use in the present compositions include those disclosed in U.S. Pat. No. 4,565,647, Llenado, issued Jan. 21, 1986. These surfactants include a hydrophobic group containing from 6 to 30 carbon atoms and a polysaccharide, e.g., a polyglycoside, hydrophilic group containing from 1.3 to 10 saccharide units. Any reducing saccharide containing 5 or 6 carbon atoms can be used, e.g., glucose, galactose and galactosyl moieties can be substituted for the glucosyl moieties. (Optionally the hydrophobic group is attached at the 2-, 3-, 4-, etc. positions thus giving a glucose or galactose as opposed to a glucoside or galactoside.) The intersaccharide bonds can be, e.g., between the one position of the additional saccharide units and the 2-, 3-, 4-, and/or 6-positions on the preceding saccharide units.

12. Fatty acid amide surfactants include those having the formula: R⁶CON(R⁷)₂ in which R⁶ is an alkyl group containing from 7 to 21 carbon atoms and each R⁷ is independently hydrogen, C₁-C₄ alkyl, C₁-C₄ hydroxyalkyl, or —(C₂H₄O)ₓH, where x is in the range of from 1 to 3.

13. A useful class of non-ionic surfactants includes the class defined as alkoxylated amines or, most particularly, alcohol alkoxylated/aminated/alkoxylated surfactants. These non-ionic surfactants may be at least in part represented by the general formulae:

R²⁰—(PO)ₛN-(EO)ₜH,

R₂0-(PO)ₛN-(EO)ₜH(EO)ₜH, and

R²⁰—N(EO)ₜH;

in which R²⁰ is an alkyl, alkenyl or other aliphatic group, or an alkyl-aryl group of from 8 to 20, preferably 12 to 14 carbon atoms, EO is oxyethylene, PO is oxypropylene, s is 1 to 20, preferably 2-5, t is 1-10, preferably 2-5, and u is 1-10, preferably 2-5. Other variations on the scope of these compounds may be represented by the alternative formula:

R²⁰—(PO)ᵥ—N[(EO)_{w}H][(EO)_{z}H]

in which R²⁰ is as defined above, v is 1 to 20 (e.g., 1, 2, 3, or 4 (preferably 2)), and w and z are independently 1-10, preferably 2-5.

These compounds are represented commercially by a line of products sold by Huntsman Chemicals as nonionic surfactants. A suitable chemical of this class includes Surfonic™. PEA 25 Amine Alkoxylate.

The treatise Nonionic Surfactants, edited by Schick, M. J., Vol. 1 of the Surfactant Science Series, Marcel Dekker, Inc., New York, 1983 is a reference on the wide variety of nonionic compounds. A typical listing of nonionic classes, and species of these surfactants, is given in U.S. Pat. No. 3,929,678. Further examples are given in “Surface Active Agents and Detergents” (Vol. I and II by Schwartz, Perry and Berch). Each of these references is herein incorporated by reference in their entirety.

Preferred nonionic surfactants include D 097 (PEG-PPG), LD 097 (Polyoxyethylene polyoxypropylene), Pluronic 25-R8 (Polyoxypropylene polyoxyethylene block), Pluronic 10R5, Neodol 45-13 (Linear C14-15 alcohol 13 mole ethoxylate), Neodol 25-12 (Linear alcohol 12 mole ethoxylate), ABIL B 9950 (Tegopren-dimethicone propyl PG), Pluronic N-3 (Propoxy-Ethoxy N-3), Novel II 1012 GB-21 (alcohol ethoxylate C10-12, 21EO), Pluronic 10R5, Pluronic 25-R2 (Polyoxypropylene polyoxyethylene block), Plurafac LF-221 (Alkoxylated Alcohol), Genapol EP-2454 (Fatty alcohol alkoxylate), Plurafac LF-500 (Alcohol ethoxylate propoxylate), Dehypon LS-36 (Ethoxylated Propoxylated Aliphatic Alcohol), Pluronic 25R2 (PO-EOOPO), and Pluronic 17R4 (polypropylene glycol)-block poly(ethylene glycol)-block-polypropylene glycol)).

### Semi Polar Nonionic Surfactants

The semi-polar type of nonionic surface-active agents is another class of useful nonionic surfactants. The semi-polar nonionic surfactants include the amine oxides, phosphine oxides, sulfoxides and their alkoxylated derivatives.

14. Amine oxides are tertiary amine oxides corresponding to the general formula:

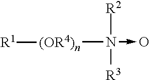

wherein the arrow is a conventional representation of a semi-polar bond; and R¹, R², and R³ may be aliphatic, aromatic, heterocyclic, alicyclic, or combinations thereof. Generally, for amine oxides of detergent interest, R¹ is an alkyl radical of from 8 to 24 carbon atoms; R² and R³ are alkyl or hydroxyalkyl of 1-3 carbon atoms or a mixture thereof R² and R³ can be attached to each other, e.g. through an oxygen or nitrogen atom, to form a ring structure; R⁴ is absent, an alkaline or a hydroxyalkylene group containing 2 to 3 carbon atoms; and n ranges from 0 to 20.

Useful water soluble amine oxide surfactants are selected from the coconut or tallow alkyl di-(lower alkyl) amine oxides, specific examples of which are dodecyldimethylamine oxide, tridecyldimethylamine oxide, tetradecyldimethylamine oxide, pentadecyldimethylamine oxide, hexadecyldimethylamine oxide, heptadecyldimethylamine oxide, octadecyldimethylamine oxide, dodecyldipropylamine oxide, tetradecyldipropylamine oxide, hexadecyldipropylamine oxide, tetradecyldibutylamine oxide, octadecyldibutylamine oxide, bis(2-hydroxyethyl) dodecylamine oxide, bis(2-hydroxyethyl)-3-dodecoxy-1-hydroxypropylamine oxide, dimethyl-(2-hydroxydodecyl)amine oxide, 3,6,9-trioctadecyldimethylamine oxide and 3-dodecoxy-2-hydroxypropyldi-(2-hydroxyethyl)amine oxide.

Useful semi-polar nonionic surfactants also include the water-soluble phosphine oxides having the following structure:

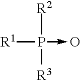

wherein the arrow is a conventional representation of a semi-polar bond; and R¹ is an alkyl, alkenyl or hydroxyalkyl moiety ranging from 10 to 24 carbon atoms in chain length; and R² and R³ are each alkyl moieties separately selected from alkyl or hydroxyalkyl groups containing 1 to 3 carbon atoms.

Examples of phosphine oxides include dimethyldecylphosphine oxide, dimethyltetradecylphosphine oxide, methylethyltetradecylphosphine oxide, dimethylhexadecylphosphine oxide, diethyl-2-hydroxyoctyldecylphosphine oxide, bis(2-hydroxyethyl) dodecylphosphine oxide, and bis(hydroxymethyl)tetradecylphosphine oxide.

Semi-polar nonionic surfactants also include the water-soluble sulfoxide compounds which have the structure:

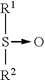

wherein the arrow is a conventional representation of a semi-polar bond; and R¹ is an alkyl or hydroxyalkyl moiety of 8 to 28 carbon atoms, from 0 to 5 ether linkages and from 0 to 2 hydroxyl substituents; and R² is an alkyl moiety consisting of alkyl and hydroxyalkyl groups having 1 to 3 carbon atoms.

Useful examples of these sulfoxides include dodecyl methyl sulfoxide; 3-hydroxy tridecyl methyl sulfoxide; 3-methoxy tridecyl methyl sulfoxide; and 3-hydroxy-4-dodecoxybutyl methyl sulfoxide.

### Anionic Surfactants

Anionic surfactants are categorized as anionics because the charge on the hydrophobe is negative; or surfactants in which the hydrophobic section of the molecule carries no charge unless the pH is elevated to neutrality or above (e.g., carboxylic acids). Carboxylate, sulfonate, sulfate and phosphate are the polar (hydrophilic) solubilizing groups found in anionic surfactants. Of the cations (counter ions) associated with these polar groups, sodium, lithium and potassium impart water solubility; ammonium and substituted ammonium ions provide both water and oil solubility; and calcium, barium, and magnesium promote oil solubility.

As those skilled in the art understand, anionics are excellent detersive surfactants and are therefore favored additions to heavy duty delimer compositions. Anionic surface-active compounds are useful to impart special chemical or physical properties other than detergency within the composition. Anionics can be employed as gelling agents or as part of a gelling or thickening system. Anionics are excellent solubilizers and can be used for hydrotropic effect and cloud point control.

The majority of large volume commercial anionic surfactants can be subdivided into five major chemical classes and additional sub-groups known to those of skill in the art and described in “Surfactant Encyclopedia,” Cosmetics & Toiletries, Vol. 104 (2) 71-86 (1989). The first class includes acylamino acids (and salts), such as acylgluamates, acyl peptides, sarcosinates (e.g., N-acyl sarcosinates), taurates (e.g., N-acyl taurates and fatty acid amides of methyl tauride), and the like. The second class includes carboxylic acids (and salts), such as alkanoic acids (and alkanoates), ester carboxylic acids (e.g., alkyl succinates), ether carboxylic acids, and the like. The third class includes phosphoric acid esters and their salts. The fourth class includes sulfonic acids (and salts), such as isethionates (e.g., acyl isethionates), alkylaryl sulfonates, alkyl sulfonates, sulfosuccinates (e.g., monoesters and diesters of sulfosuccinate), and the like. The fifth class includes sulfuric acid esters (and salts), such as alkyl ether sulfates, alkyl sulfates, and the like.

Anionic sulfate surfactants include the linear and branched primary and secondary alkyl sulfates, alkyl ethoxysulfates, fatty oleyl glycerol sulfates, alkyl phenol ethylene oxide ether sulfates, the C₅-C₁₇ acyl-N—(C₁-C₄ alkyl) and —N—(C₁-C₂ hydroxyalkyl) glucamine sulfates, and sulfates of alkylpolysaccharides such as the sulfates of alkylpolyglucoside (the nonionic nonsulfated compounds being described herein).

Examples of suitable synthetic, water soluble anionic detergent compounds include the ammonium and substituted ammonium (such as mono-, di- and triethanolamine) and alkali metal (such as sodium, lithium and potassium) salts of the alkyl mononuclear aromatic sulfonates such as the alkyl benzene sulfonates containing from 5 to 18 carbon atoms in the alkyl group in a straight or branched chain, e.g., the salts of alkyl benzene sulfonates or of alkyl toluene, xylene, cumene and phenol sulfonates; alkyl naphthalene sulfonate, diamyl naphthalene sulfonate, and dinonyl naphthalene sulfonate and alkoxylated derivatives.

Anionic carboxylate surfactants include the alkyl ethoxy carboxylates, the alkyl polyethoxy polycarboxylate surfactants and the soaps (e.g., alkyl carboxyls). Secondary soap surfactants (e.g., alkyl carboxyl surfactants) include those which contain a carboxyl unit connected to a secondary carbon. The secondary carbon can be in a ring structure, e.g., as in p-octyl benzoic acid, or as in alkyl-substituted cyclohexyl carboxylates. The secondary soap surfactants typically contain no ether linkages, no ester linkages and no hydroxyl groups. Further, they typically lack nitrogen atoms in the head-group (amphiphilic portion). Suitable secondary soap surfactants typically contain 11-13 total carbon atoms, although more carbons atoms (e.g., up to 16) can be present.

Other anionic surfactants include olefin sulfonates, such as long chain alkene sulfonates, long chain hydroxyalkane sulfonates or mixtures of alkenesulfonates and hydroxyalkane-sulfonates. Also included are the alkyl sulfates, alkyl poly(ethylencoxy) ether sulfates and aromatic poly(ethyleneoxy) sulfates such as the sulfates or condensation products of ethylene oxide and nonyl phenol (usually having 1 to 6 oxyethylene groups per molecule). Resin acids and hydrogenated resin acids are also suitable, such as rosin, hydrogenated rosin, and resin acids and hydrogenated resin acids present in or derived from tallow oil.

The particular salts will be suitably selected depending upon the particular formulation and the needs therein.

Further examples of suitable anionic surfactants are given in “Surface Active Agents and Detergents” (Vol. I and II by Schwartz, Perry and Berch), which is herein incorporated by reference in its entirety. A variety of such surfactants are also generally disclosed in U.S. Pat. No. 3,929,678 at Column 23, line 58 through Column 29, line 23.

### Amphoteric Surfactants

Amphoteric, or ampholytic, surfactants contain both a basic and an acidic hydrophilic group and an organic hydrophobic group. These ionic entities may be any of the anionic or cationic groups described herein for other types of surfactants. A basic nitrogen and an acidic carboxylate group are the typical functional groups employed as the basic and acidic hydrophilic groups. In a few surfactants, sulfonate, sulfate, phosphonate or phosphate provide the negative charge.

Amphoteric surfactants can be broadly described as derivatives of aliphatic secondary and tertiary amines, in which the aliphatic radical may be straight chain or branched and wherein one of the aliphatic substituents contains from 8 to 18 carbon atoms and one contains an anionic water solubilizing group, e.g., carboxy, sulfo, sulfato, phosphato, or phosphono. Amphoteric surfactants are subdivided into two major classes known to those of skill in the art and described in “Surfactant Encyclopedia,” Cosmetics & Toiletries, Vol. 104 (2) 69-71 (1989), which is herein incorporated by reference in its entirety. The first class includes acyl/dialkyl ethylenediamine derivatives (e.g., 2-alkyl hydroxyethyl imidazoline derivatives) and their salts. The second class includes N-alkylamino acids and their salts. Some amphoteric surfactants can be envisioned as fitting into both classes.

Amphoteric surfactants can be synthesized by methods known to those of skill in the art. For example, 2-alkyl hydroxyethyl imidazoline is synthesized by condensation and ring closure of a long chain carboxylic acid (or a derivative) with dialkyl ethylenediamine. Commercial amphoteric surfactants are derivatized by subsequent hydrolysis and ring-opening of the imidazoline ring by alkylation—for example with ethyl acetate. During alkylation, one or two carboxy-alkyl groups react to form a tertiary amine and an ether linkage with differing alkylating agents yielding different tertiary amines.

Long chain imidazole derivatives generally have the general formula:

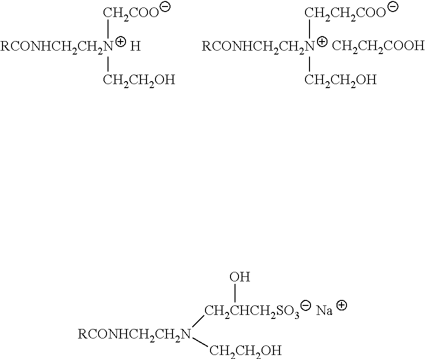

wherein R is an acyclic hydrophobic group containing from 8 to 18 carbon atoms and M is a cation to neutralize the charge of the anion, generally sodium. Commercially prominent imidazoline-derived amphoterics include for example: Cocoamphopropionate, Cocoamphocarboxy-propionate, Cocoamphoglycinate, Cocoamphocarboxy-glycinate, Cocoamphopropyl-sulfonate, and Cocoamphocarboxy-propionic acid. Preferred amphocarboxylic acids are produced from fatty imidazolines in which the dicarboxylic acid functionality of the amphodicarboxylic acid is diacetic acid and/or dipropionic acid.

The carboxymethylated compounds (glycinates) described herein above frequently are called betaines. Betaines are a special class of amphoteric discussed herein below in the section entitled, Zwitterion Surfactants.

Long chain N-alkylamino acids are readily prepared by reacting RNH₂, in which R is a C₈-C₁₈ straight or branched chain alkyl, fatty amines with halogenated carboxylic acids. Alkylation of the primary amino groups of an amino acid leads to secondary and tertiary amines. Alkyl substituents may have additional amino groups that provide more than one reactive nitrogen center. Most commercial N-alkylamine acids are alkyl derivatives of beta-alanine or beta-N(2-carboxyethyl) alanine. Examples of commercial N-alkylamino acid ampholytes include alkyl beta-amino dipropionates, RN(C₂H₄COOM)₂ and RNHC₂H₄COOM. In these, R is preferably an acyclic hydrophobic group containing from 8 to 18 carbon atoms, and M is a cation to neutralize the charge of the anion.

Preferred amphoteric surfactants include those derived from coconut products such as coconut oil or coconut fatty acid. The more suitable of these coconut derived surfactants include as part of their structure an ethylenediamine moiety, an alkanolamide moiety, an amino acid moiety, preferably glycine, or a combination thereof; and an aliphatic substituent of from 8 to 18 (preferably 12) carbon atoms. Such a surfactant can also be considered an alkyl amphodicarboxylic acid. Disodium cocoampho dipropionate is one most suitable amphoteric surfactant and is commercially available under the tradename Miranol™ FBS from Rhodia Inc., Cranbury, N.J. Another most suitable coconut derived amphoteric surfactant with the chemical name disodium cocoampho diacetate is sold under the tradename Miranol™ C₂M-SF Conc., also from Rhodia Inc., Cranbury, N.J.

A typical listing of amphoteric classes, and species of these surfactants, is given in U.S. Pat. No. 3,929,678 issued to Laughlin and Heuring on Dec. 30, 1975. Further examples are given in “Surface Active Agents and Detergents” (Vol. I and II by Schwartz, Perry and Berch), which is herein incorporated by reference in its entirety.

### Zwitterionic Surfactants

Zwitterionic surfactants can be thought of as a subset of the amphoteric surfactants. Zwitterionic surfactants can be broadly described as derivatives of secondary and tertiary amines, derivatives of heterocyclic secondary and tertiary amines, or derivatives of quaternary ammonium, quaternary phosphonium or tertiary sulfonium compounds. Typically, a zwitterionic surfactant includes a positive charged quaternary ammonium or, in some cases, a sulfonium or phosphonium ion, a negative charged carboxyl group, and an alkyl group. Zwitterionics generally contain cationic and anionic groups which ionize to a nearly equal degree in the isoelectric region of the molecule, and which can develop strong “inner-salt” attraction between positive-negative charge centers. Examples of such zwitterionic synthetic surfactants include derivatives of aliphatic quaternary ammonium, phosphonium, and sulfonium compounds, in which the aliphatic radicals can be straight chain or branched, and wherein one of the aliphatic substituents contains from 8 to 18 carbon atoms and one contains an anionic water solubilizing group, e.g., carboxy, sulfonate, sulfate, phosphate, or phosphonate. Betaine and sultaine surfactants are exemplary zwitterionic surfactants for use herein.

A general formula for these compounds is:

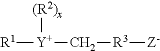

wherein R¹ contains an alkyl, alkenyl, or hydroxyalkyl radical of from 8 to 18 carbon atoms having from 0 to 10 ethylene oxide moieties and from 0 to 1 glyceryl moiety; Y is selected from the group consisting of nitrogen, phosphorus, and sulfur atoms; R² is an alkyl or monohydroxy alkyl group containing 1 to 3 carbon atoms; x is 1 when Y is a sulfur atom and 2 when Y is a nitrogen or phosphorus atom, R³ is an alkylene or hydroxy alkylene or hydroxy alkylene of from 1 to 4 carbon atoms and Z is a radical selected from the group consisting of carboxylate, sulfonate, sulfate, phosphonate, and phosphate groups.

Examples of zwitterionic surfactants having the structures listed above include: 4-[N,N-di(2-hydroxyethyl)-N-octadecylammonio]-butane-1-carboxylate; 5-[S-3-hydroxypropyl-S-hexadecylsulfonio]-3-hydroxypentane-1-sulfate; 3-[P,P-diethyl-P-3,6,9-trioxatetracosanephosphonio]-2-hydroxypropane 1-phosphate; 3-[N,N-dipropyl-N-3-dodecoxy-2-hydroxypropyl-ammonio]-propane-1-phosphonate; 3-(N,N-dimethyl-N-hexadecylammonio)-propane-1-sulfonate; 3-(N,N-dimethyl-N-hexadecylammonio)-2-hydroxy-propane-1-sulfonate; 4-[N,N-di (2 (2-hydroxyethyl)-N(2-hydroxydodecyl) ammonio]-butane-1-carboxylate; 3-[S-ethyl-S-(3-dodecoxy-2-hydroxypropy) sulfonio]-propane-1-phosphate; 3-[P,P-dimethyl-P-dodecylphosphonio]-propane-1-phosphonate; and S [N,N-di(3-hydroxypropyl)-N-hexadecylammonio]-2-hydroxy-pentane-1-sulfate. The alkyl groups contained in said detergent surfactants can be straight or branched and saturated or unsaturated.

The zwitterionic surfactant suitable for use in the present compositions includes a betaine of the general structure:

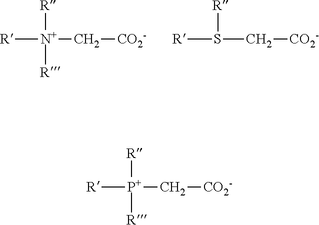

These surfactant betaines typically do not exhibit strong cationic or anionic characters at pH extremes, nor do they show reduced water solubility in their isoelectric range. Unlike “external” quaternary ammonium salts, betaines are compatible with anionics. Examples of suitable betaines include coconut acylamidopropyldimethyl betaine; hexadecyl dimethyl betaine; C₁₂₋₁₄ acylamidopropylbetaine; C₈₋₁₄ acylamidohexyldiethyl betaine; 4-C₁₄₋₁₆ acylmethylamidodiethylammonio-1-carboxybutane; C₁₆₋₁₈ acylamidodimethylbetaine; C₁₂₋₁₆ acylamidopentanediethylbetaine; and C₁₂₋₁₆ acylmethylamidodimethylbetaine.

Sultaines include those compounds having the formula (R(R¹)₂N⁺R²SO³⁻, in which R is a C₆-C₁₈ hydrocarbyl group, each R¹ is typically independently C₁-C₃ alkyl, e.g., methyl, and R² is a C₁-C₆ hydrocarbyl group, e.g., a C₁-C₃ alkylene or hydroxyalkylene group.

A typical listing of zwitterionic classes, and species of these surfactants, is given in U.S. Pat. No. 3,929,678 issued to Laughlin and Heuring on Dec. 30, 1975. Further examples are given in “Surface Active Agents and Detergents” (Vol. I and II by Schwartz, Perry and Berch), which is herein incorporated by reference in its entirety.

### Alkylpolyglucoside

In some embodiments the compositions optionally include one or more alkylpolysaccharides, particularly alkylpolyglucosides. Examples of suitable alkyl polysaccharides are alkyl polyglucosides having the formula:

R²O(CₙH₂ₙO)ₜ(Z)ₓ
wherein Z is derived from glucose, R² is a hydrophobic group such as an alkyl, alkyl phenyl, hydroxyalkyl, hydroxyalkylphenyl group, or a combination thereof, in which said alkyl groups contain from about 10 to about 18, preferably from 12 to 16 carbon atoms; n is 2-6, t is from 0 to about 10; and x is from 0 to about 10, preferably from 1 to 4, most preferably from 1.4.

Preferred alkyl polyglycosides are alkyl polyglycosides having the formula:

R₁O(R₂O)_{b}(Z)ₐ
wherein Z is glucose or a glucose residue and b is zero. Such alkyl polyglycosides are commercially available, for example, as Glucopon® or Plantaren® surfactants from Henkel Corporation. Examples of such surfactants include but are not limited to Glucopon® 225 or Glucopon® 215, an alkyl polyglycoside in which the alkyl group contains 8 to 10 carbon atoms and has an average degree of polymerization of 1.7; Glucopon® 425, an alkyl polyglycoside in which the alkyl group contains 8 to 16 carbon atoms and has an average degree of polymerization of 1.6; Glucopon® 625, an alkyl polyglycoside in which the alkyl group contains 12 to 16 carbon atoms and has an average degree of polymerization of 1.6; APG® 325, an alkyl polyglycoside in which the alkyl group contains 8 to 12 carbon atoms and has an average degree of polymerization of 1.6; Glucopon® 600, an alkyl polyglycoside in which the alkyl group contains 12 to 16 carbon atoms and has an average degree of polymerization of 1.4; Plantaren® 2000, a C₈-C₁₈ alkyl polyglycoside in which the alkyl group contains 8 to 16 carbon atoms and has an average degree of polymerization of 1.4; Plantaren® 1300, a C₁₂-C₁₆ alkyl polyglycoside in which the alkyl group contains 12 to 16 carbon atoms and has an average degree of polymerization of 1.6; and combinations thereof.

### Additional Functional Ingredients

The components of the antimicrobial compositions can optionally be further combined with various additional functional components. In some embodiments, the compositions including the at least one acid, alkyl ether carboxylic acid, water, and optional additional surfactant(s) make up a large amount, or even substantially all of the total weight of the composition. For example, in some embodiments few or no additional functional ingredients are included therein.

In other embodiments, additional functional ingredients may be included in the compositions. The functional ingredients provide desired properties and functionalities to the compositions. For the purpose of this application, the term “functional ingredient” includes a material that when dispersed or dissolved in a use and/or concentrate solution, such as an aqueous solution, provides a beneficial property in a particular use. Some particular examples of functional materials are discussed in more detail below, although the particular materials discussed are given by way of example only, and that a broad variety of other functional ingredients may be used.

In preferred embodiments, the compositions do not include quaternary ammonium compounds. In additional embodiments, the compositions do not include conventional Norovirus actives, including for example, ethanol, silver citrate, and/or electrolytic chlorine. In additional embodiments the compositions do not include alcohols and/or other organic solvents to beneficially provide a non-flammable product. In additional embodiments the compositions do not include fatty acid esters. In still further additional embodiments, the compositions do not include hydrogen peroxide and/or peroxycarboxylic acids. In further embodiments the compositions do not include any of quaternary ammonium compounds, conventional Norovirus actives, alcohols and/or other organic solvents, fatty acid esters, hydrogen peroxide and/or peroxycarboxylic acids.

In other embodiments, the compositions may include defoaming agents, wetting agents, anti-redeposition agents, solubility modifiers, dispersants, rinse aids, metal protecting agents, stabilizing agents, corrosion inhibitors, sequestrants and/or chelating agents, threshold agent, additional surfactants, fatty acid esters, fragrances and/or dyes, rheology modifiers or thickeners, hydrotropes or couplers, buffers, solvents, sensor indicators, and the like.

In embodiments the compositions include one or more additional functional ingredients in the composition from about 0 wt. % to about 90 wt. %, from about 0 wt. % to about 50 wt. %, or from about 0 wt. % to about 30 wt. %, from about 0 wt. % to about 25 wt. %.

In embodiments the compositions include one or more additional functional ingredients in the composition from about 0.01 wt. % to about 90 wt. %, from about 0.01 wt. % to about 30 wt. %, from about 0.01 wt. % to about 25 wt. %.

### Fatty Acid Esters

The antimicrobial compositions can optionally include fatty acid esters. A fatty acid ester (FAE) is an ester resulting from the combination of a fatty acid with an alcohol.

Examples of fatty acid esters can include sulfonated fatty acid esters, C6-C24 fatty acid ester ethoxylates, propoxylates or glycerides, or preferably C8-C12 fatty acid ester ethoxylates, propoxylates or glycerides. An example of a C6-C24, namely a C8-C10 fatty acid ester glycerides include octanoic/decanoic esters of glycerol. Still further examples of fatty acid esters can include glycerides (including monoglycerides, diglycerides, and triglycerides) when the alcohol is glycerol, sorbitan fatty acid esters (i.e. sorbitan sugar esters including for example sorbitan monooleate, sorbitan monooleatepolyoxyethylene ether), sorbitol fatty acid esters, polyethylene glycol fatty acid esters (e.g. tall oil fatty acids), polyglycerol fatty acid esters and the like.

Commercially available fatty acid esters include for example, Stepan GCC-Mild, Stepan 108, Tween 20, etc. For example, polyoxyethylene sorbitan fatty acid esters can be Tween 20, Tween 40, Tween 60 and Tween 80, while the sorbitan fatty acid esters can be Span 20, Span 40, Span 60 and Span 80.

If included in the compositions, the compositions include from about 1 wt. % to about 30 wt. % of the fatty acid esters, from about 1 wt. % to about 20 wt. % of the fatty acid esters, from about 5 wt. % to about 20 wt. % of the fatty acid esters, or from about 5 wt. % to about 15 wt. % of the fatty acid esters. In addition, without being limited according to the disclosure, all ranges recited are inclusive of the numbers defining the range and include each integer within the defined range.

### Defoaming Agents

Defoaming agents can optionally be included in the compositions. Generally, defoamers which can be used include alcohol alkoxylates and EO/PO block copolymers. Defoamers can also include polyalkylene glycol condensates and propyl glycols, including polypropyl glycol. In some embodiments, the compositions can include antifoaming agents or defoamers which are of food grade quality given the application of the methods. To this end, one of the more effective antifoaming agents includes silicones. Silicones such as dimethyl silicone, glycol polysiloxane, methylphenol polysiloxane, trialkyl or tetralkyl silanes, hydrophobic silica defoamers and mixtures thereof can all be used in defoaming applications.

Defoamers can be present at a concentration range from about 0.01 wt. % to 20 wt. %, 0.01 wt. % to 20 wt. %, from about 0.01 wt. % to 5 wt. %, or from about 0.01 wt. % to about 1 wt. %.

### Methods of Use

The antimicrobial compositions are particularly well suited for treating surfaces in need of antimicrobial efficacy against a broad spectrum of microorganisms. In further aspects, the antimicrobial compositions with excellent antimicrobial activity are still further well suited for treating surfaces in need of virucidal efficacy against small, non-enveloped viruses, large, non-enveloped viruses and/or any enveloped viruses without the use of any quaternary ammonium compounds and/or sulfonated anionic surfactants having regulatory limitations.

The methods of use for antimicrobial, including antiviral, disinfection, include a contacting step, wherein the composition is applied to a surface in need of treatment. In an aspect, contacting the composition is to a surface contaminated with a microorganism, including any one of bacteria, virus, fungus, or a combination thereof. The contaminated surfaces can be precleaned and/or soiled.

In a preferred aspect, the methods of use provide complete kill of a Norovirus. Beneficially, in an aspect, greater than a 99.9% reduction (3-log order reduction) in such population, greater than 99.99% reduction (4-log order reduction) in such populations, or greater than a 99.999% reduction (5-log order reduction) in the population of a Norovirus on a surface is achieved with a contact time of less than about 60 minutes, less than about 30 minutes, less than about 15 minutes, less than about 5 minutes, less than about 1 minute, less than about 30 seconds, or even less than about 15 seconds.

In a further aspect, contacting the antimicrobial compositions can be to a food contact and/or non-food contact hard surface. Such surfaces can further include instruments, such as medical instruments. Surfaces can also include those cleaned in third-sink sanitizing, including various wares. In still further aspects, contacting the composition can be to a CIP (clean in place) application.

In still further aspects, contacting the antimicrobial compositions can be to a ware wash machine, such as a ware wash application.

In still further aspects, contacting the antimicrobial compositions can be to a third sink sanitizing application or first sink disinfecting detergent application. In a still further aspect, the contacting is beneficially compatible with first sink detergents, such that a third sink sanitizing step could be used as a water recycle to combine with a first sink detergent. This is a benefit over conventional compositions containing quaternary ammonium compounds which are not compatible with first sink detergents.

In still further aspects, contacting the antimicrobial compositions can be to a tissue surface, including tissue treatment applications. Example tissue surfaces include mammalian skin, such as animal or human skin, including for example human hands.

The various surfaces to which the antimicrobial compositions can be applied can include any conventional application means. Application can include, for example, by wiping, spraying, dipping, immersing, or the like. The contacting can also include providing a solid to be first dissolved in water to form a solution for the contacting. The contacting step allows the composition to contact the soiled surface for a predetermined amount of time. The amount of time can be sufficient to allow, including from a few seconds to an hour, from about 15 seconds, or about 30 seconds to about 60 minutes, or any range therebetween. In a preferred embodiment, the contact time required for antimicrobial efficacy is less than about 30 minutes, less than about 15 minutes, less than about 10 minutes, less than about 5 minutes, or less than about 1 minute. In still further embodiments, the contact time required for antimicrobial efficacy is less than about 30 seconds, or even less than about 15 seconds.

The methods provide benefits of composition stability and efficacy performance using hard water sources as a diluent to form a use solution and/or using under hard water conditions. Hard water includes dissolved solids such as calcium and magnesium. In general “hard water” is high in calcium and magnesium salts relative to “soft water” where the levels of these minerals are much lower. In an embodiment hard water can be measured by containing at least about 100 ppm calcium carbonate. In many hard water conditions the molar ratio of calcium to magnesium in hard water is about 2:1 or about 3:1. However, hard water measurements vary by location.

Hard water further exacerbates streaking that can be caused by anionic surfactant containing antimicrobial compositions. Beneficially, the compositions comprising, consisting of, or consisting essentially of carboxylic acid(s), alkyl sulfate surfactant and sulfonate surfactants do not cause streaking on treated hard surfaces, beneficially eliminating the need to rinse after an application of the composition to a surface.

The methods may comprise a single step of applying the antimicrobial compositions onto the surface without direct physical removal, such as a rinse step and/or a wiping step. Beneficially, in various embodiments the compositions can optionally provide a no-rinse application. A no rinse application is particularly beneficial for spray compositions as no additional potable water source for a rinse is required due to efficacy of the compositions at use ppm concentration ranges. As a further benefit, in various embodiments the antimicrobial compositions can optionally provide a wiping application. As a still further benefit, in various embodiments the antimicrobial compositions can provide a no-rinse and no wiping application. In some aspects, the methods of use include contacting an antimicrobial composition with a surface at a use concentration between about 0.2% to about 2% (active basis), or from about 0.25% to about 1.50% (active basis).

In some aspects, the methods can further include a precleaning step, such as where a cleaning compositions is applied, wiped and/or rinsed, and thereafter followed by the applying of the antimicrobial compositions. The compositions and methods of use thereof can include treating cleaned or soiled surfaces. In some embodiments the amount of contact time between the composition and the surface is sufficient to reduce the population of microorganisms (including fungi, bacteria and/or viral pathogens) on a surface to provide greater than a 99.9% reduction (3-log order reduction) in such population, greater than 99.99% reduction (4-log order reduction) in such populations, or greater than a 99.999% reduction (5-log order reduction) in the population of microorganisms and pathogens. The contact time is preferably less than about 30 minutes, less than about 15 minutes or less than about 5 minutes.

Temperature conditions for the methods can range from about 40° F.-160° F., about 60° F.-140° F., or about 70° F.-140° F.

Beneficially, the methods do not require a rinse step. In an aspect, the antimicrobial compositions are food contact approved and do not require a rinse step. As a further benefit, the methods do not cause corrosion and/or interfere with surfaces (e.g., hazy, dull or other negative aesthetic effects on the surface).

The methods can optionally include the use of various sensors and/or indicators. In an aspect, the level of active ingredients in use solution can be monitored by various ways. In one approach, the critical pH of the solution at which the product will start to lose its biocidal efficacy significantly is visually indicated by a color change, and the color change is achieved by choosing a dye that show dramatic color change at this pH. The dye could be simply incorporated into the product, and preferably the dye is incorporated into a polymeric substrate to form a color change strip, and the strip will put in the container, for example the third sink to show the color change when the solution pass the critical pH value. Additionally, the level of anionic surfactants in use solution could also be monitored by a similar manner, where a color change will indicate the critical concentration of anionic surfactant needed for biocidal efficacy.

In an additional embodiment, as an alternative to visual indicators, properties of the use solution including pH, anionic activity, fluorescence, and/or conductivity can be monitored by sensors that provide a visual or audible signal when the solution is no longer within a specified range. In some embodiments, a marker molecule can be added to the composition, where the change of the active ingredients in the use solution will trigger the physical and/or chemical property changes of the marker molecule, and the change is quantified through a signal processing.

In embodiments, the antimicrobial compositions meet bactericidal requirements for EN1276 (bacterial suspension study), EN13697 (bacteria-carrier based study), and EN1650 (bacterial suspension study) at 18° C.-25° C. under clean and/or dirty conditions. In embodiments, the antimicrobial compositions meet virucidal requirements for EN14476 at 18° C.-25° C. under clean and/or dirty conditions. As one skilled in the art will appreciate, the suspension studies can also be referred to as Phase 2, Step 1 (or 2.1-suspension) studies and the carrier studies can also be referred to as Phase 2, Step 2 (or 2.2-carrier) studies or also laboratory simulated surface tests.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

It is to be understood that while the disclosure has been described in conjunction with the detailed description thereof, the foregoing description is intended to illustrate, and not limit the scope of the disclosure, which is defined by the scope of the appended claims. Other embodiments, advantages, and modifications are within the scope of the following claims. Any reference to accompanying drawings which form a part hereof, are shown, by way of illustration only. It is understood that other embodiments may be utilized, and structural changes may be made without departing from the scope of the present disclosure. All publications discussed and/or referenced herein are incorporated herein in their entirety.

### Example 1. Antimicrobial Performance

The formulations of Table 2 were used for evaluation of various anionic surfactants for efficacy against bacterial populations according to AOAC Method 960.09. Test organisms included E. coli and S. aureus. Test formulations were prepared as shown in Table 2, were added into a flask containing a suspension of the test microorganisms, and then allowed to interact for a 30 second exposure. At the end of the contact time, a small portion of the product and test suspension was neutralized to ascertain the number of surviving microorganisms. The results are shown in Table 3. In this Table, “TNTC” refers to “too numerous to count.”

TABLE 2

| | Formula | Formula | Formula | Formula | Formula | Formula | Formula |
|---|---|---|---|---|---|---|---|
| | 1 | 2 | 3 | 4 | 5 | 6 | 7 |
| Component | wt. % | wt. % | wt. % | wt. % | wt. % | wt. % | wt. % |
| LAS (96%) | 17.32 | 0 | 0.00 | 5.56 | 17.32 | 0.00 | 0.00 |
| SLS (30%) | 0.00 | 30.00 | 17.78 | 0.00 | 0.00 | 30.00 | 30.00 |
| AOS (40%) | 0.00 | 0.00 | 11.11 | 22.22 | 0.00 | 0.00 | 0.00 |
| AOS (solid) | 0.00 | 0.00 | 0.00 | 0.00 | 0.00 | 12.50 | 7.50 |
| Lactic acid | 38.00 | 40.50 | 0.00 | 0.00 | 30.50 | 40.50 | 40.50 |
| (88%) | | | | | | | |
| Citric acid | 39.70 | 17.00 | 53.33 | 53.33 | 39.70 | 17.00 | 17.00 |
| (50%) | | | | | | | |
| EO/PO | 5.00 | 0.00 | 0.00 | 0.00 | 12.50 | 0.00 | 0.00 |
| Copolymer | | | | | | | |
| C10-C12 | 0.00 | 12.50 | 0.00 | 0.00 | 0.00 | 0.00 | 0.00 |
| Alcohol | | | | | | | |
| Ethoxylate | | | | | | | |
| C8-C10 | 0.00 | 0.00 | 5.47 | 0.00 | 0.00 | 0.00 | 5.00 |
| Alkylpoly | | | | | | | |
| glucoside | | | | | | | |
| DI water | 0.00 | 0.00 | 12.31 | 18.89 | 0.00 | 0.00 | 0.00 |

TABLE 3

| | | | | | | | | Log |
|---|---|---|---|---|---|---|---|---|
| | | Exposure | CFU - Dilution | | | | | Reduction |
| Sample | pH | Temp | 1E−01 | 1E−02 | 1E−03 | CFU/mL | Log | (E. coli) |
| Formula 1 | 2.61 | 10° C. | TNTC | 228 | 19 | 2.2E+04 | 4.35 | 3.49 |
| (0.39%) | | | | | | | | |
| Formula 2 | 2.90 | 10° C. | TNTC | TNTC | TNTC | 3.0E+05 | 5.48 | 2.37 |
| (0.39%) | | | | | | | | |
| Formula 3 | 2.86 | 25° C. | 0 | 0 | 0 | 1.0E+01 | 1.00 | 6.81 |
| (0.59%) | | | | | | | | |
| Formula 3 | 2.86 | 10° C. | TNTC | TNTC | TNTC | 3.0E+05 | 5.48 | 2.37 |
| (0.59%) | | | | | | | | |
| Formula 3 | 2.82 | 10° C. | TNTC | TNTC | 330 | 3.3E+05 | 5.52 | 2.33 |
| (0.65%) | | | | | | | | |
| Formula 4 | 2.76 | 25° C. | 0 | 0 | 0 | 1.0E+01 | 1.00 | 6.81 |
| (0.59%) | | | | | | | | |
| Formula 4 | 2.76 | 10° C. | TNTC | TNTC | TNTC | 3.0E+05 | 5.48 | 2.37 |
| (0.59%) | | | | | | | | |
| Formula 4 | 2.70 | 10° C. | TNTC | TNTC | TNTC | 3.0E+05 | 5.48 | 2.37 |
| (1.25%) | | | | | | | | |
| Formula 5 | 2.63 | 25° C. | 0 | 0 | 0 | 1.0E+01 | 1.00 | 6.81 |
| (0.39%) | | | | | | | | |
| Formula 5 | 2.63 | 10° C. | TNTC | TNTC | 39 | 3.9E+04 | 4.59 | 3.25 |
| (0.39%) | | | | | | | | |
| Formula 5 | 2.59 | 10° C. | TNTC | 24 | 3 | 2.5E+03 | 3.39 | 4.46 |
| (0.41%) | | | | | | | | |
| Formula 6 | 2.87 | 25° C. | 0 | 0 | 0 | 1.0E+01 | 1.00 | 6.81 |
| (0.39%) | | | | | | | | |
| Formula 6 | 2.87 | 10° C. | TNTC | TNTC | TNTC | 3.0E+05 | 5.48 | 2.37 |
| (0.39%) | | | | | | | | |
| Formula 7 | 2.88 | 25° C. | 0 | 0 | 0 | 1.0E+01 | 1.00 | 6.81 |
| (0.39%) | | | | | | | | |
| Formula 7 | 2.88 | 10° C. | TNTC | TNTC | TNTC | 3.0E+05 | 5.48 | 2.37 |
| (0.39%) | | | | | | | | |
| Formula 1 | 2.61 | 10° C. | TNTC | 228 | 19 | 2.2E+04 | 4.35 | 3.49 |
| (0.39%) | | | | | | | | |
| Formula 2 | 2.90 | 10° C. | TNTC | TNTC | TNTC | 3.0E+05 | 5.48 | 2.37 |
| (0.39%) | | | | | | | | |
| Formula 3 | 2.86 | 25° C. | 0 | 0 | 0 | 1.0E+01 | 1.00 | 6.81 |
| (0.59%) | | | | | | | | |
| Formula 3 | 2.86 | 10° C. | TNTC | TNTC | TNTC | 3.0E+05 | 5.48 | 2.37 |
| (0.59%) | | | | | | | | |
| Formula 3 | 2.82 | 10° C. | TNTC | TNTC | 330 | 3.3E+05 | 5.52 | 2.33 |
| (0.65%) | | | | | | | | |
| Formula 4 | 2.76 | 25° C. | 0 | 0 | 0 | 1.0E+01 | 1.00 | 6.81 |
| (0.59%) | | | | | | | | |
| Formula 4 | 2.76 | 10° C. | TNTC | TNTC | TNTC | 3.0E+05 | 5.48 | 2.37 |
| (0.59%) | | | | | | | | |
| Formula 4 | 2.70 | 10° C. | TNTC | TNTC | TNTC | 3.0E+05 | 5.48 | 2.37 |
| (1.25%) | | | | | | | | |
| Formula 5 | 2.63 | 25° C. | 0 | 0 | 0 | 1.0E+01 | 1.00 | 6.81 |
| (0.39%) | | | | | | | | |
| Formula 5 | 2.63 | 10° C. | TNTC | TNTC | 39 | 3.9E+04 | 4.59 | 3.25 |
| (0.39%) | | | | | | | | |
| Formula 5 | 2.59 | 10° C. | TNTC | 24 | 3 | 2.5E+03 | 3.39 | 4.46 |
| (0.41%) | | | | | | | | |
| Formula 6 | 2.87 | 25° C. | 0 | 0 | 0 | 1.0E+01 | 1.00 | 6.81 |
| (0.39%) | | | | | | | | |
| Formula 6 | 2.87 | 10° C. | TNTC | TNTC | TNTC | 3.0E+05 | 5.48 | 2.37 |
| (0.39%) | | | | | | | | |
| Formula 7 | 2.88 | 25° C. | 0 | 0 | 0 | 1.0E+01 | 1.00 | 6.81 |
| (0.39%) | | | | | | | | |
| Formula 7 | 2.88 | 10° C. | TNTC | TNTC | TNTC | 3.0E+05 | 5.48 | 2.37 |
| (0.39%) | | | | | | | | |

### Example 2. Bactericidal Efficacy Screening

Bactericidal efficacy screening was conducted with the chemistries of Table 4 following AOAC Method 960.09 as described in Example 1 using S. aureus (ATCC 6538), E. coli (ATCC 11229), L. monocytogenes (ATCC 19117), S. enterica (ATCC 10708) and E. coli 0157: H7 (ATCC 35150), at a 30-second contact time. The testing was conducted at 25±1° C. with a 30 second contact time using 500 ppm AOAC Synthetic hard water (SHW) and 17 Grain tap water (HW) as the diluent sources. The results are shown in Table 5.

TABLE 4

| | Formula A | Formula B | Formula C | Formula D |
|---|---|---|---|---|
| Component | wt. % | wt. % | wt. % | wt. % |
| SLS (30%) | 29.00 | 29.00 | 24.00 | 24.00 |
| AOS (40%) | 18.75 | 18.75 | 15.00 | 15.00 |
| Lactic acid (88%) | 32.19 | 42.46 | 32.19 | 42.46 |
| Citric acid (50%) | 13.60 | 0.00 | 13.60 | 0.00 |
| APG 215 (65%) | 5.00 | 5.00 | 4.00 | 4.00 |
| DI water | 1.46 | 4.79 | 11.21 | 14.54 |

TABLE 5

| | Test | | | | | | |
|---|---|---|---|---|---|---|---|
| Test | Formula | | CFU - Dilution | | | | Log |
| Organism | (0.39%) | Diluent | 1E−01 | 1E−02 | CFU/mL | Log | Reduction |
| E. coli | Formula A | | 0 | 0 | 1.0E+01 | 1.00 | 6.85 |
| | Formula B | 500 | 0 | 0 | 1.0E+01 | 1.00 | 6.85 |
| | Formula C | ppm | 0 | 0 | 1.0E+01 | 1.00 | 6.85 |
| | Formula D | SHW | 0 | 0 | 1.0E+01 | 1.00 | 6.85 |
| | Formula A | 17 | TNTC | 43 | 4.3E+03 | 3.63 | 4.22 |
| | Formula B | Grain | 6 | 1 | 6.4E+01 | 1.80 | 6.05 |
| | Formula C | HW | 121 | 18 | 1.3E+03 | 3.10 | 4.75 |
| | Formula D | | 6 | 0 | 5.5E+01 | 1.74 | 6.11 |
| S. aureus | Formula A | 500 | 0 | 0 | 1.0E+01 | 1.00 | 6.72 |
| | Formula B | ppm | 0 | 0 | 1.0E+01 | 1.00 | 6.72 |
| | Formula C | SHW | 0 | 0 | 1.0E+01 | 1.00 | 6.72 |
| | Formula D | | 0 | 0 | 1.0E+01 | 1.00 | 6.72 |
| | Formula A | 17 | 0 | 0 | 1.0E+01 | 1.00 | 6.72 |
| | Formula B | Grain | 0 | 0 | 1.0E+01 | 1.00 | 6.72 |
| | Formula C | HW | 0 | 0 | 1.0E+01 | 1.00 | 6.72 |
| | Formula D | | 0 | 0 | 1.0E+01 | 1.00 | 6.72 |
| Listeria | Formula A | 500 | 0 | 0 | 1.0E+01 | 1.00 | 7.10 |
| | Formula B | ppm | 0 | 0 | 1.0E+01 | 1.00 | 7.10 |
| | Formula C | SHW | 0 | 0 | 1.0E+01 | 1.00 | 7.10 |
| | Formula D | | 0 | 0 | 1.0E+01 | 1.00 | 7.10 |
| | Formula A | 17 | 0 | 0 | 1.0E+01 | 1.00 | 7.10 |
| | Formula B | Grain | 0 | 0 | 1.0E+01 | 1.00 | 7.10 |
| | Formula C | HW | 0 | 0 | 1.0E+01 | 1.00 | 7.10 |
| | Formula D | | 0 | 0 | 1.0E+01 | 1.00 | 7.10 |
| Salmonella | Formula A | 500 | 0 | 0 | 1.0E+01 | 1.00 | 7.05 |
| | Formula B | ppm | 0 | 0 | 1.0E+01 | 1.00 | 7.05 |
| | Formula C | SHW | 0 | 0 | 1.0E+01 | 1.00 | 7.05 |
| | Formula D | | 0 | 0 | 1.0E+01 | 1.00 | 7.05 |
| | Formula A | 17 | 0 | 0 | 1.0E+01 | 1.00 | 7.05 |
| | Formula B | Grain | 2 | 0 | 1.8E+01 | 1.26 | 6.79 |
| | Formula C | HW | 32 | 3 | 3.2E+02 | 2.50 | 5.55 |
| | Formula D | | 6 | 1 | 6.4E+01 | 1.80 | 6.25 |
| E. coli | Formula A | 500 | 0 | 0 | 1.0E+01 | 1.00 | 7.04 |
| O157:H7 | Formula B | ppm | 0 | 0 | 1.0E+01 | 1.00 | 7.04 |
| | Formula C | SHW | 0 | 0 | 1.0E+01 | 1.00 | 7.04 |
| | Formula D | | 0 | 0 | 1.0E+01 | 1.00 | 7.04 |
| | Formula A | 17 | 49 | 2 | 4.6E+02 | 2.67 | 5.38 |
| | Formula B | Grain | 5 | 1 | 5.5E+01 | 1.74 | 6.30 |
| | Formula C | HW | 160 | 18 | 1.6E+03 | 3.21 | 4.83 |
| | Formula D | | 22 | 4 | 2.4E+02 | 2.37 | 5.67 |

### Example 3. Use Solution Assessment

Using the methodologies described herein, base formulations were prepared according to Table 6. These compositions were diluted in water and evaluated for their antimicrobial efficacy against Feline Calcivirus. The results are shown into Table 7. Additional example use solutions were prepared and are shown in Tables 8-11.

TABLE 6

| Component | Formula 1 | Formula 2 | AOS1 | AOS2 | AOS/LAS1 | AOS/LAS2 |
|---|---|---|---|---|---|---|
| LAS | 17.30 | | | | 6.94 | 6.94 |
| SLS | | 30.00 | | | | |
| AOS | | | 33.33 | 33.33 | 16.67 | 16.67 |
| Lactic acid | 38.00 | 40.50 | | | | |
| Citric acid | 39.70 | 17.00 | 42.67 | 66.67 | 53.33 | 65.72 |
| EO/PO | 5.00 | | | | | |
| block | | | | | | |
| copolymer | | | | | | |
| Alcohol | | 12.50 | | | | |
| Ethoxylate | | | | | | |
| Alkylpoly | | | | | 10.67 | 10.67 |
| glucoside | | | | | | |
| DI water | | | 24.00 | 0.00 | 12.39 | 0.00 |
| Total | 100 | 100 | 100 | 100 | 100 | 100 |

TABLE 7

| | | | Average | | |
|---|---|---|---|---|---|
| | | | Control | Test | Log |
| | | Test | Result | Result | Reduc- |
| Product ID | Product Diluent | Organisms | (log10) | (log10) | tion |
| 0.5 oz/gal | 500 ppm AOAC | Feline | 6 | 0.75 | 5.25 |
| AOS-2 | synthetic hard | Calicivirus | | | |
| | water | | | | |
| 0.5 oz/gal | 500 ppm AOAC | Feline | 6 | ≤0.50 | ≥5.50 |
| AOS/LAS-2 | synthetic hard | Calicivirus | | | |
| | water | | | | |
| 0.5 oz/gal | 500 ppm AOAC | Feline | 6 | ≤0.50 | ≥5.50 |
| Formula 1 | synthetic hard | Calicivirus | | | |
| | water | | | | |
| 0.5 oz/gal | 500 ppm AOAC | Feline | 6 | ≤0.50 | ≥5.50 |
| Formula 2 | synthetic hard | Calicivirus | | | |
| | water | | | | |
| 0.5 oz/gal | 500 ppm AOAC | Feline | 6 | 4.75 | 1.25 |
| Formula 2 | synthetic hard | Calicivirus | | | |
| | water | | | | |

TABLE 8

| | Exp | Exp | Exp | Exp | Exp | Exp | Exp | Exp | Exp |
|---|---|---|---|---|---|---|---|---|---|
| Component | 1 | 2 | 3 | 4 | 5 | 6 | 7 | 8 | 9 |
| Anionic Surfactant | ppm | ppm | ppm | ppm | ppm | ppm | ppm | ppm | ppm |
| Linear Alkyl Benzene | 500 | 500 | 500 | 500 | 500 | 500 | 500 | 500 | 500 |
| Sulfonic Acid (LAS) | | | | | | | | | |
| Alkyl Olefin Sulfonate | | | | | | | | | |
| (AOS) | | | | | | | | | |
| Sodium Lauryl Sulfate | | | | | | | | | |
| (SLS) | | | | | | | | | |
| C8-C10 | | 100 | 200 | 300 | 400 | | | | |
| Alkylpolyglucoside | | | | | | | | | |
| C8-C18 | | | | | | 100 | 200 | 300 | 400 |
| Alkylpolyglucoside | | | | | | | | | |
| 17gpg water | | | | | | | | | |
| Citric Acid | 1000 | 1000 | 1000 | 1000 | 1000 | 1000 | 1000 | 1000 | 1000 |

TABLE 9

| | Exp | Exp | Exp | Exp | Exp | Exp | Exp | Exp | Exp | Exp |
|---|---|---|---|---|---|---|---|---|---|---|
| Component | 10 | 11 | 12 | 13 | 14 | 15 | 16 | 17 | 18 | 19 |
| Anionic Surfactant | ppm | ppm | ppm | ppm | ppm | ppm | ppm | ppm | ppm | ppm |
| Linear Alkyl | | | | | | | | | | |
| Benzene Sulfonic | | | | | | | | | | |
| Acid (LAS) | | | | | | | | | | |
| Alkyl Olefin | 500 | | | | | | | | | |
| Sulfonate (AOS) | | | | | | | | | | |
| Sodium Lauryl | | 500 | 500 | 500 | 500 | 500 | 500 | 500 | 500 | 500 |
| Sulfate (SLS) | | | | | | | | | | |
| C8-C10 Alkylpoly | | | 100 | 200 | 300 | 400 | | | | |
| glucoside | | | | | | | | | | |
| C8-C18 Alkylpoly | | | | | | | 100 | 200 | 300 | 400 |
| glucoside | | | | | | | | | | |
| 17gpg water | | | | | | | | | | |
| Citric Acid | 1000 | 1000 | 1000 | 1000 | 1000 | 1000 | 1000 | 1000 | 1000 | 1000 |

TABLE 10

| | Exp | Exp | Exp | Exp | Exp | Exp | Exp | Exp | Exp |
|---|---|---|---|---|---|---|---|---|---|
| Component | 20 | 21 | 22 | 23 | 24 | 25 | 26 | 27 | 28 |
| Anionic Surfactant | ppm | ppm | ppm | ppm | ppm | ppm | ppm | ppm | ppm |
| Linear Alkyl | 250 | 250 | 250 | 250 | 250 | 250 | 250 | 250 | 250 |
| Benzene Sulfonic | | | | | | | | | |
| Acid (LAS) | | | | | | | | | |
| Alkyl Olefin | 250 | 250 | 250 | 250 | 250 | 250 | 250 | 250 | 250 |
| Sulfonate (AOS) | | | | | | | | | |
| Sodium Lauryl | | | | | | | | | |
| Sulfate (SLS) | | | | | | | | | |
| C8-C10 | | 100 | 200 | 300 | 400 | | | | |
| Alkylpolyglucoside | | | | | | | | | |
| C8-C18 | | | | | | 100 | 200 | 300 | 400 |
| Alkylpolyglucoside | | | | | | | | | |
| 17gpg water | | | | | | | | | |
| Citric Acid | 1000 | 1000 | 1000 | 1000 | 1000 | 1000 | 1000 | 1000 | 1000 |

TABLE 11

| | Exp | Exp | Exp | Exp | Exp | Exp | Exp |
|---|---|---|---|---|---|---|---|
| Component | 38 | 39 | 40 | 41 | 42 | 43 | 44 |
| Anionic Surfactant | ppm | ppm | ppm | ppm | ppm | ppm | ppm |
| Linear Alkyl Benzene Sulfonic | 300 | 350 | 400 | 450 | 500 | 550 | 600 |
| Acid (LAS) | | | | | | | |
| Alkyl Olefin Sulfonate (AOS) | 250 | 250 | 250 | 250 | 250 | 250 | 250 |
| Sodium Lauryl Sulfate (SLS) | | | | | | | |
| C8-C10 Alkylpolyglucoside | | | | | | | |
| C8-C18 Alkylpolyglucoside | | | | | | | |
| 17gpg water | | | | | | | |
| Citric Acid | 1000 | 1000 | 1000 | 1000 | 1000 | 1000 | 1000 |

### Example 4

Example formulations were prepared as shown in Table 12. This composition was evaluated for hardwater tolerance and streaking using 500 ppm AOAC Synthetic hard water (SHW) and 17 Grain tap water (HW) as the diluent sources. The results are shown in Tables 13-14.

TABLE 12

| Component | Formula E (wt. %) |
|---|---|
| SLS (30%) | 28 |
| AOS (40%) | 18.5 |
| Lactic acid (88%) | 49 |
| Citric acid (50%) | 4.5 |
| Total | 100 |

TABLE 13

| Hardwater Tolerance 17 gpg | Formula E |
|---|---|
| 1 hr | Clear, stable solution |
| 6 hr | Clear, stable solution |
| 24 hr | Clear, stable solution |

TABLE 14
Steaking Tests - Evaluation conducted
on glass - Visual assessment.

| | | Commercial | | |
|---|---|---|---|---|
| | Commercial | Product A | Formula E | Formula E |
| | Product B | 4 oz/gal | @ 0.2% | @ 0.39% |
| DI water | quite streaky | spotty | pretty good | spotty |
| 0 gpg | quite streaky | very spotty | pretty good | pretty good |
| 5 gpg | quite streaky | very spotty | pretty good | pretty good |

### Example 5

Example formulations were prepared as shown in Table 15 comparing single carboxylic acid composition with a combination carboxylic acid combination with the sulfate and sulfonate surfactants. The compositions were evaluated for microbial efficacy under hard water conditions 500 ppm AOAC Synthetic hard water (SHW) and 17 Grain tap water (HW) as the diluent sources. The results are shown in Table 16.

TABLE 15

| Component | Formula F (wt. %) | Formula G (wt. %) |
|---|---|---|
| SLS (30%) | 28 | 28 |
| AOS (40%) | 18.5 | 18.5 |
| Lactic acid (88%) | 48 | 48 |
| Nonanoic acid | 0 | 2.5 |
| DI water | 5.5 | 3 |
| Total | 100 | 100 |

TABLE 16

| | | Contact | Microorganism | |
|---|---|---|---|---|
| Composition | Concentration | Time (min) | Staph | Pseudo |
| F | 0.40% | 5.00 | 58/60 | 60/60 |
| | 0.40% | 5.00 | 59/60 | |
| | 0.40% | 5.00 | 58/60 | |
| | 0.45% | 5.00 | 58/60 | |
| | 0.50% | 5.00 | 59/60 | |
| | 0.55% | 5.00 | 60/60 | |
| | 0.60% | 5.00 | 60/60 | |
| G | 0.40% | 5.00 | 60/60 | |
| | 0.40% | 5.00 | 60/60 | |
| | 0.40% | 3.00 | 59/60 | 60/60 |

The results in Table 16 show Formula F with a single carboxylic acid over increasing concentrations did not pass micro efficacy testing at lower concentrations (<0.55%) whereas the Formula G combining a first and second carboxylic acid, namely a hydroxyacids and fatty acid, passed micro efficacy testing at a 0.4% concentration.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the disclosure in diverse forms thereof.

All publications and patent applications in this specification are indicative of the level of ordinary skill in the art to which this disclosure pertains. All publications and patent applications are herein incorporated by reference to the same extent as if each individual publication or patent application were specifically and individually indicated as incorporated by reference.

1. An antimicrobial composition comprising:
at least one carboxylic acid;
an alkyl sulfate; and
a sulfonate.

2. The composition of claim 1, wherein the carboxylic acid comprises at least one hydroxy acid and/or polycarboxylic acid.

3. The composition of claim 2, further comprising a second carboxylic acid comprising a fatty acid.

4. The composition of claim 1, wherein the carboxylic acid is selected from the group consisting of acetic acid, formic acid, benzoic acid, propionic acid, octanoic acid, nonanoic acid, decanoic acid, palmitic acid, stearic acid, oleic acid, lactic acid, citric acid, malic acid, tartaric acid, succinic acid, oxalic acid and combinations thereof.

5. The composition of claim 1, further comprising an additional acid.

6. The composition of claim 1, wherein the alkyl sulfate comprises a C8-C18 alkyl sulfate.

7. The composition of claim 1, wherein the alkyl sulfate comprises sodium lauryl sulfate (SLS), sodium laureth sulfate (SLES), ammonium lauryl sulfate (ALS), or a combination thereof.

8. The composition of claim 1, wherein the sulfonate comprises an alpha olefin sulfonate, alkane sulfonate, alkyl sulfonate, sulfonated carboxylic acid ester, or a combination thereof.

9. The composition of claim 1, wherein the sulfonate comprises an alpha olefin sulfonate.

10. The composition of claim 9, wherein the alkyl sulfate is a C8-C18 alkyl sulfate and the sulfonate is a C8-C22 alpha olefin sulfonate, and the sulfonate is a C8-C22 or a C8-C16 alpha olefin sulfonate.

11. The composition of claim 1, wherein the composition further comprises an additional functional ingredient comprising a nonionic surfactant, alkylpolyglucoside, additional anionic surfactant, cationic surfactant, or a combination thereof, and/or wherein the composition is free of quaternary ammonium compounds, Norovirus actives, alcohols and/or other organic solvents, fatty acid esters, hydrogen peroxide and/or peroxycarboxylic acids.

12. The composition of claim 1, wherein the composition comprises;
(a) from about 5 wt. % to about 35 wt. % of the alkyl sulfate; from about 10 wt. % to about 35 wt. % of the sulfonate; and from about 30 wt. % to about 60 wt. % of the carboxylic acid(s) or from about 30 wt. % to about 60 wt. % of a first carboxylic acid and from about 0 wt. % to about 10 wt. % of a second carboxylic acid;
(b) from about 15 wt. % to about 35 wt. % of the alkyl sulfate; from about 15 wt. % to about 30 wt. % of the sulfonate; from about 35 wt. % to about 55 wt. % of the carboxylic acid or from about 35 wt. % to about 55 wt. % of a first carboxylic acid and from about 0 wt. % to about 8 wt. % of a second carboxylic acid;
(c) from about 25 wt. % to about 35 wt. % of the alkyl sulfate; from about 15 wt. % to about 25 wt. % of the sulfonate; from about 35 wt. % to about 50 wt. % of the carboxylic acid or from about 35 wt. % to about 50 wt. % of a first carboxylic acid and from about 1 wt. % to about 5 wt. % of a second carboxylic acid; and/or
(d) at least about 40 wt. % of the combined alkyl sulfate and the sulfonate.

13. A method of using an antimicrobial composition comprising:
contacting the antimicrobial composition of claim 1 with a surface at a use concentration between about 0.2% to about 20% (active basis); and
providing at least a 3-log reduction of a microbial population, and wherein the antimicrobial composition is hard water tolerant and does not streak on the surface.

14. The method of claim 13, wherein the composition is diluted to form a use solution before the contacting.

15. The method of claim 13, wherein the at least 3-log reduction occurs within 10 minutes of the contacting.

16. The method of claim 13, wherein the contacting is by wiping, dipping, immersing, or spraying.

17. The method of claim 13, wherein the antimicrobial composition is contacted with a surface at a use concentration between about 0.25% to about 1.50% (active basis).

18. The method of claim 13, wherein the surface is a hard surface.

19. The method of claim 13, wherein the contacting step is at an aqueous use temperature from about 20° C. to about 71° C.

20. The method of claim 13, wherein the method does not require a rinse step before the contacting, after the contacting, or a combination thereof.
